(19) United States
(12) Patent Application Publication
(10) Pub. No.: US 2011/0305673 A1
(43) Pub. Date: Dec. 15, 2011

(54) COMPOSITIONS AND METHODS FOR TISSUE REPAIR

(75) Inventor: Jeffrey Spees, Colchester, VT (US)

(73) Assignee: The University of Vermont and State Agriculture College, Burlington, VT (US)

(21) Appl. No.: 13/128,804

(22) PCT Filed: Nov. 12, 2009
(86) PCT No.: PCT/US09/06099

§ 371 Date: Aug. 31, 2011

Related U.S. Application Data
(60) Provisional application No. 61/113,842, filed on Nov. 12, 2008.

(51) Int. Cl. A61K 35/28; C12Q 1/02; A61P 43/00; C12N 5/071

(52) U.S. Cl. 424/93.7; 435/325; 424/577; 435/375; 435/29

(57) ABSTRACT

The invention features compositions comprising mesenchymal stem cells or multipotent stromal cells, agents secreted by such cells in culture, and methods featuring such cells for the repair or regeneration of a damaged tissue or organ. The present invention is based, at least in part, on the discovery that agents secreted by bone marrow mesenchymal stem cells or multipotent stromal cells (MSCs) were useful for the treatment or prevention of tissue damage related to ischemic injury (e.g., cerebral or cardiac ischemia).

### CROSS-REFERENCE TO RELATED APPLICATION

This application claims the benefit of the following U.S. Provisional Application No. 61/113,842, which was filed on Nov. 12, 2008.

### STATEMENT OF RIGHTS TO INVENTIONS MADE UNDER FEDERAL SPONSORED RESEARCH

This work was supported by the following grants from the National Institutes of Health, Grant Nos: HL085210 NIH/NHLBI (JLS) and P20 RR016435 NIH/NCRR. The government has certain rights in the invention.

### BACKGROUND OF THE INVENTION

All mammalian cells require a consistent source of oxygen to allow them to function normally. When their access to oxygen is interrupted, cell damage and death can quickly result. Certain cell types, including muscle cells and neurons are particularly vulnerable to ischemic injury in connection with myocardial infarction and stroke. Despite recent advances in treating ischemic injuries, stroke and myocardial infarction continue to kill or disable vast numbers of people each year. In the United States alone, about 780,000 people experience a new or recurrent stroke annually. Of those American who do survive, many will experience serious long term disability. In the United States alone, 600,000 new myocardial infarctions and 320,000 recurrent attacks occur annually. About 38 percent of the people who experience a myocardial infarction in a given year will die, while many of those who survive will experience some loss in cardiac function. Accordingly, improved methods of treating tissue injury, particularly ischemic injuries associated with stroke and myocardiac infarction are urgently required.

### SUMMARY OF THE INVENTION

As described below, the present invention features compositions and methods for promoting tissue repair.

In one aspect, the invention generally provides a cellular composition containing an isolated bone marrow-derived cell or an in vitro-derived progeny cell thereof that expresses CD133 or CD271/p75-low affinity nerve growth factor receptor. In one embodiment, at least about 50% (e.g., 50, 60, 70, 80, 90 or 100%) of the cells present in the composition express CD133 or CD271/p75-low affinity nerve growth factor receptor or are derived from a CD133 or CD271/p75-low affinity nerve growth factor receptor progenitor cell. In one embodiment, the cellular composition contains isolated cells that have not yet been passaged. In another embodiment, the cellular composition contains cells cultured for at least two, three, four, five or more passages. In one embodiment, the expression of CD133 or CD271/p75-low affinity nerve growth factor receptor, which were originally used to isolate the cells, is no longer detectable or is reduced during the course of the passages. In another embodiment, the cellular composition contains one or more cells that express one or more surface epitopes that is CD133⁺, CD45⁺, CD34⁺, ABC G2⁺, or CD24⁺, and fails to express detectable levels or express reduced levels of a surface epitope selected from the group consisting of CD49a, CD49b, CD90, and CD105. In yet another embodiment, the cellular composition contains cells that at passage 2 fail to express detectable levels or express reduced levels of a surface epitope selected from the group consisting of CD133, CD45, CD34, CD31, ABCG2 or CD24. In still another embodiment, the cellular composition contains cells that express a surface epitope selected from the group consisting of CD90 (Thy 1), CD105 (Endoglin), CD29, CD44, CD59, CD49a and CD49b. In still another embodiment, the cellular composition contains cells that express increased levels CD146. In still another embodiment, the cells are capable of differentiating into osteoblasts, adipocytes, and chondrocytes.

In another aspect, the invention provides a composition contains secreted cellular factors in a pharmaceutical excipient, where the cellular factors are derived from a cell of the previous aspect or otherwise delineated herein.

In another aspect, the composition contains secreted cellular factors in a pharmaceutical excipient, where the cellular factors are greater than about 5 kD is size; detectable in an immunoassay; secreted by an isolated bone marrow-derived non-hematopoietic progenitor cell selected for expression of CD133 or CD271/p75-low affinity nerve growth factor receptor;

have a biological activity that is any one or more of reducing cell death in a cell population at risk thereof, increasing cell survival, reducing inflammation, increase cell proliferation; and/or inactivated by heat denaturation.

In yet another aspect, the invention provides a method for generating a composition that promotes tissue repair, the method involves selecting an isolated bone marrow-derived cell that expresses CD133 or CD271/p75-low affinity nerve growth factor receptor; and incubating the cell in growth media to enrich the media for cell-secreted factors, thereby generating a composition that promotes tissue repair. In one embodiment, the method further involves purifying the cell-secreted factors. In another embodiment, the purification involves selecting fractions having a desired biological activity. In still another embodiment, the selected fraction increases cell survival, reduces cell death, increases cell proliferation, or increases tissue or organ function. In still another embodiment, the selected fraction lacks an undesirable biological activity that is any one or more of reducing cell survival, increasing cell death, and reducing cell proliferation. In one embodiment, the cell is a cell in vitro or a non-human cell in vivo. In another embodiment, the cell is a mesenchymal stem cell or multipotent stromal cell.

In another aspect, the method for increasing cell survival or proliferation involves obtaining a composition according to the previous aspect, and contacting a cell at risk of cell death with the composition, thereby increasing cell survival or proliferation.

In another aspect, the invention provides a method for stabilizing or reducing tissue damage in a subject, the method involving obtaining a composition according to a method of a previous aspect or otherwise delineated herein, and contacting a cell of the subject with an effective amount of the composition, thereby stabilizing or reducing tissue damage in the subject.

In still another aspect, the invention provides a method for increasing cell survival or proliferation, the method involving contacting a cell with an effective amount of a composition containing factors secreted by an isolated bone marrow-derived cell that expresses CD133 or CD271/p75-low affinity nerve growth factor receptor; thereby increasing cell survival or proliferation.

In still another aspect, the invention provides a method for stabilizing or reducing tissue or organ damage in a subject, the method involving administering to the subject an effective amount of a composition containing factors secreted by an isolated bone marrow-derived cell that expresses CD133 or CD271/p75-low affinity nerve growth factor receptor, thereby stabilizing or reducing tissue or organ damage. In one embodiment, the administering increases the cell number or biological function of the tissue or organ. In another embodiment, the method increases the number of cells of the tissue or organ by at least about 5% compared to a corresponding untreated control tissue or organ. In still another embodiment, the method increases the biological activity of the tissue or organ by at least about 5% compared to a corresponding untreated control tissue or organ. In still another embodiment, the composition is administered directly to a site of tissue damage or disease. In still another embodiment, the composition is administered systemically. In still another embodiment, the subject has a disease selected from the group consisting of myocardial infarction, congestive heart failure, stroke, ischemia, and wound healing. In still another embodiment, the method improves motor function after stroke or improves heart function after an ischemic event relative to the subject's function prior to treatment or relative to a reference. In still another embodiment, the method reduces infarct volumes, reduces cell death, or protects against cerebral ischemia.

In still another aspect, the invention provides a subject-specific cellular composition for increasing cell survival or proliferation, the cellular composition containing a bone marrow-derived cell from the subject, where the cell expresses CD133 or CD271/p75-low affinity nerve growth factor receptor and an excipient.

In still another aspect, the invention provides a subject-specific composition containing secreted cellular factors in a pharmaceutical excipient, where the cellular factors are secreted by a bone marrow-derived cell from the subject, where the cell expresses CD133 or CD271/p75-low affinity nerve growth factor receptor. In one embodiment, at least about 50% of the cells express CD133 or CD271/p75-low affinity nerve growth factor receptor or are derived from a CD133 or CD271/p75-low affinity nerve growth factor receptor progenitor cell. In another embodiment, the cellular composition contains cells cultured for at least two passages. In still another embodiment, the cellular composition contains cells that express one or more surface epitopes selected from the group consisting of CD133⁺, CD45⁺, CD34⁺, ABC G2⁺, CD24⁺, and fail to express detectable levels or express reduced levels of a surface epitope selected from the group consisting of CD49a, CD49b, CD90, and CD105. In still another embodiment, the cellular composition contains cells that at passage 2 fail to express detectable levels or express reduced levels of a surface epitope selected from the group consisting of CD133, CD45, CD34, CD31, ABCG2 or CD24. In still another embodiment, the cellular composition contains cells that express a surface epitope selected from the group consisting of CD90 (Thy 1), CD105 (Endoglin), CD29, CD44, CD59, CD49a and CD49b. In another embodiment, the cellular composition contains cells that express increased levels CD146. In another embodiment, the composition further contains cryoprotectants.

In yet another aspect, the invention provides a method for increasing cell survival or proliferation in a subject, the method involving contacting a cell with an effective amount of a composition containing factors secreted by an isolated bone marrow-derived cell that expresses CD133 or CD271/p75-low affinity nerve growth factor receptor or a cell of any one of claims 21-29; thereby increasing cell survival or proliferation.

In yet another aspect, the invention provides a method for treating or preventing ischemic damage in a subject, the method involving contacting a cell at risk of ischemic injury with an effective amount of a composition containing factors secreted by an isolated bone marrow-derived cell that expresses CD133 or CD271/p75-low affinity nerve growth factor receptor or a cell of any one of claims 21-29; thereby increasing cell survival or proliferation. In one embodiment, the factors are derived from a cell is isolated from the subject. In another embodiment, the factors are frozen prior to administration to the subject. In one embodiment, the method prevents or ameliorates ischemic damage or reduces apoptosis or increases cell proliferation. In another embodiment, the cell is a neural or muscle stem cell or progenitor cell. In another embodiment, the cell is present in a tissue or organ. In another embodiment, the tissue is cardiac tissue or neural tissue. In still another embodiment, the method repairs or prevents post-infarct ischemic damage in a cardiac tissue. In still another embodiment, the method repairs hind limb ischemia in a skeletal muscle tissue. In still another embodiment, the method increases biological function following an ischemic injury relative to the biological function of an untreated control tissue.

In another aspect, the invention provides a method of ameliorating tissue damage in a subject, the method involving obtaining a non-hematopoietic stem cell from the subject; isolating factors secreted by the stem cell; storing the factors; and contacting a cell in need thereof of the subject thereby ameliorating a cardiovascular condition.

In still another aspect, the invention provides a method of ameliorating a cardiovascular condition in a subject, the method involving obtaining a non-hematopoietic stem cell from the subject; isolating factors secreted by the stem cell; storing the factors; and contacting a cardiac cell of the subject thereby ameliorating a cardiovascular condition. In one embodiment, the method increases left ventricular function, reduces fibrosis, or increases myocite survival in a cardiac tissue of the subject.

In still another aspect, the invention provides a method of ameliorating a neuronal damage related to ischemia in a subject, the method involving obtaining a non-hematopoietic stem cell from the subject; isolating factors secreted by the stem cell; storing the factors; and contacting a neuronal cell of the subject with the factor thereby ameliorating neuronal damage related to ischemia.

In various embodiments of the above aspects, the method further involves expressing a recombinant protein (e.g., a polypeptide that promotes cell proliferation or reduces cell death) in the cell.

In still another aspect, the invention provides a method for identifying an agent useful for tissue repair or regeneration, the method involving contacting a cell or cell population at risk of cell death with a composition of agents secreted by an isolated bone marrow-derived non-hematopoietic progenitor cell selected for expression of CD133 and/or CD271/p75-low affinity nerve growth factor receptor; detecting an increase in cell survival, growth, or proliferation or a decrease in cell death relative to an untreated control cell or cell population. identifying an agent or fraction of the composition that reduces cell death, increases cell growth or proliferation.

The invention provides compositions and methods for promoting tissue repair, for reducing cell death, and for reducing inflammation. The invention further provides agents that are useful for the development of highly specific drugs for use in tissue repair or to treat or a disorder characterized by the methods delineated herein. In addition, the methods of the invention provide a facile means to identify therapies that are safe for use in eukaryotic host organisms. In addition, the methods of the invention provide a route for analyzing virtually any number of compounds for effects on a disease described herein with high-volume throughput, high sensitivity, and low complexity.

Other features and advantages of the invention will be apparent from the detailed description, and from the claims.

### DEFINITIONS

By “cellular composition” is meant any composition comprising one or more isolated cells.

By “CD133” is meant a polypeptide that binds an antibody generated against the CD133 antigen. An exemplary sequence of a CD133 antigen is provided at NCBI Accession No. AAM33415, which is reproduced below.

| | |
|---|---|
| 1 | malvlgslll lglcgnsfsg gqpsstdapk awnyelpatn |
| | yetqdshkag pigilfelvh |
| 61 | iflyvvqprd fpedtlrkfl qkayeskidy dkivyyeagi |
| | ilccvlgllf iilmplvgyf |
| 121 | fcmcrccnkc ggemhqrqke ngpflrkcfa isllviciii |
| | sigifygfva nhqvrtrikr |
| 181 | srkladsnfk dlrtllnetp eqikyilaqy nttkdkaftd |
| | lnsinsvlgg gildrlrpni |
| 241 | ipvldeiksm ataiketkea lenmnstlks lhqqstqlss |
| | sltsvktslr sslndplclv |
| 301 | hpssetcnsi rlslsqlnsn pelrqlppvd aeldnvnnvl |
| | rtdldglvqq gyqslndipd |
| 361 | rvqrqtttvv agikrvlnsi gsdidnvtqr lpiqdilsaf |
| | svyvnntesy ihrnlptlee |
| 421 | ydsywwlggl vicslltliv ifyylgllcg vcgydrhatp |
| | ttrgcvsntg gvflmvgvgl |
| 481 | sflfcwilmi ivvltfvfga nveklicepy tskelfrvld |
| | tpyllnedwe yylsgklfnk |
| 541 | skmkltfeqv ysdckknrgt ygtlhlqnsf nisehlnine |
| | htgsissele slkvnlnifl |
| 601 | lgaagrknlq dfaacgidrm nydsylaqtg kspagvnlls |
| | faydleakan slppgnlrns |
| 661 | lkrdaqtikt ihqqrvlpie qslstlyqsv kilqrtgngl |
| | lervtrilas ldfaqnfitn |
| 721 | ntssviieet kkygrtiigy fehylqwief sisekvasck |
| | pvataldtav dvflcsyiid |
| 781 | pinlfwfgig katvfllpal ifavklakyy rrmdsedvyd |
| | dvetipmknm engnngyhkd |
| 841 | hvygihnpvm tspsqh |

An exemplary CD133 polypeptide is described by Singh et al., Identification of a cancer stem cell in human brain tumors. Cancer Res. 63: 5821-5828, 2003.

By “CD271/p75-low affinity nerve growth factor receptor” is meant a polypeptide that binds nerve growth factor with low affinity or that binds an antibody generated against the p75-low affinity growth factor receptor. An exemplary sequence of p75-low affinity nerve growth factor receptor is provided at NCBI Accession No. NP_{—}002498, which is reproduced below.

| | |
|---|---|
| 1 | mgagatgram dgprllllll lgvslggake acptglyths |
| | gecckacnlg egvaqpcgan |
| 61 | qtvcepclds vtfsdvvsat epckpctecv glqsmsapcv |
| | eaddavcrca ygyyqdettg |
| 121 | rceacrvcea gsglvfscqd kqntvceecp dgtysdeanh |
| | vdpclpctvc edterqlrec |
| 181 | trwadaecee ipgrwitrst ppegsdstap stqepeappe |
| | qdliastvag vvttvmgssq |
| 241 | pvvtrgttdn lipvycsila avvvglvayi afkrwnsckq |
| | nkqgansrpv nqtpppegek |
| 301 | lhsdsgisvd sqslhdqqph tqtasgqalk gdgglysslp |
| | pakreevekl lngsagdtwr |
| 361 | hlagelgyqp ehidsfthea cpvrallasw atqdsatlda |
| | llaalrriqr adlveslcse |
| 421 | statspv |

By “cell survival” is meant cell viability.

By “detectable levels” is meant that the amount of an analyte is sufficient for detection using methods routinely used to carry out such an analysis.

By “passage” is meant the number of times a culture of cells has been split into one or more cultures to provide for continued cell survival or proliferation.

By “mesenchymal stem cell” or “multipotent stromal cell” is meant a cell of mesodermal origin or a cell capable of giving rise to progeny cells that are or give rise to connective tissue cells, bone cells, cartilage cells, cells of the circulatory system, or cells of the lymphatic systems.

By “reducing inflammation” is meant reducing cytokine secretion, white blood cell influx to an area, swelling, heat, redness, pain, or any other indication of inflammation known in the art.

By “reducing cell death” is meant reducing the propensity or probability that a cell will die. Cell death can be apoptotic, necrotic, or by any other means.

By “reduced level” is meant that the amount of an analyte in a sample is lower than the amount of the analyte in a corresponding control sample.

By “secreted cellular factor” is meant any biologically active agent that a cell secretes during in vitro culture.

By “surface epitope” is meant the expression of an antigen on the membrane of a cell.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “deficiency of a particular cell-type” is meant fewer of a specific set of cells than are normally present in a tissue or organ not having a deficiency. For example, a deficiency is a 5%, 10%, 15%, 20%, 30%, 40%, 50%, 60%, 70%, 80%, 90%, or even 100% deficit in the number of cells of a particular cell-type (e.g., adipocytes, endothelial cells, endothelial precursor cells, fibroblasts, cardiomyocytes, neurons) relative to the number of cells present in a naturally-occurring, corresponding tissue or organ. Methods for assaying cell-number are standard in the art, and are described in (Bonifacino et al., Current Protocols in Cell Biology, Loose-leaf, John Wiley and Sons, Inc., San Francisco, Calif., 1999; Robinson et al., Current Protocols in Cytometry Loose-leaf, John Wiley and Sons, Inc., San Francisco, Calif., October 1997).

“Derived from” as used herein refers to the process of obtaining a cell from a subject, embryo, biological sample, or cell culture.

“Detect” refers to identifying the presence, absence or amount of the object to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include any disease or injury that results in a reduction in cell number or biological function, including ischemic injury, such as stroke, myocardial infarction, or any other ischemic event that causes tissue damage, peripheral vascular disease, wounds, burns, fractures, blunt trauma, arthritis, and inflammatory diseases.

By “effective amount” is meant the amount of a required to ameliorate the symptoms of a disease relative to an untreated patient. The effective amount of active compound(s) used to practice the present invention for therapeutic treatment of a ischemic injury varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or, disorder.

As used herein, “obtaining” as in “obtaining an agent” includes synthesizing, purchasing, or otherwise acquiring the agent.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or there between.

By “repair” is meant to ameliorate damage or disease in a tissue or organ.

By “tissue” is meant a collection of cells having a similar morphology and function.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

### BRIEF DESCRIPTION OF THE DRAWINGS

FIGS. 1A and 1B show results of FACS analysis for cell surface epitopes. FIG. 1A illustrates changes in cell surface epitope expression that occur in freshly-isolated CD133-positive cells from human bone marrow (CD133 BM) before and after they adhere to generate CD133dMSCs. Over time, the CD133 BM cells lose the expression of CD133 and acquire the expression of typical adherent hMSC markers such as CD90 (Thy 1) and CD105 (endoglin). Passage 2 (P2) CD133dMSCs, p75dMSCs, and hMSCs are negative for CD34 and the pan-hematopoietic marker CD45. Antibody staining is shown in dark fill and isotype staining is shown in white fill. FIG. 1B shows a summary for all epitopes tested. Two donors were stained for each cell type. ND=not determined. +=1-25% cells positive, ++=25-50% cells positive, +++=50-100% cells positive. The following abbreviations are used throughout the drawings. CD133dMSCs: CD133-derived multipotent stromal cells. p75dMSCs: p75-derived multipotent stromal cells. hMSCs: human multipotent stromal cells.

FIGS. 2A-2I are photomicrographs showing the multipotent differentiation of CD133dMSCs and p75dMSCs. FIGS. 2A-2C are phase contrast photomicrographs of cultured hMSCs, CD133dMSCs, and p75dMSCs (10×). FIGS. 2D-2F show the differentiation of CD133dMSCs into osteoblasts (10×), adipocytes (10×), and chondrocytes (4×), respectively. FIGS. 2G-2I show the differentiation of p75dMSCs into osteoblasts (10×), adipocytes (40×), and chondrocytes (40×), respectively. Calcification is stained by Alizerin Red S. Lipid is stained by Oil Red O, Sulfated proteoglycans are stained by Toluidine blue sodium borate.

FIG. 3 shows the growth of hMSCs, CD133dMSCs and p75dMSCs under normoxic and hypoxic conditions. hMSCs isolated by typical plastic adherence as well as those isolated by MACS against CD133 or p75LNGFR grow equally well under normoxic and hypoxic (1% oxygen) conditions. Cell growth data are shown for 2 donors for each cell type over 8 days. Cells from all of the donors were plated at 100 cells/cm² and allowed to grow for 2 days in a normoxic incubator prior to moving half of the plates to a hypoxic incubator to begin the assay (day 0).

FIG. 4 shows the microarray analysis of expressed genes. FIG. 4 (top panel) shows hierarchical clustering for gene expression for CD133-positive and p75LNGFR-positive cells freshly isolated from human bone marrow mononuclear cells and passage 2 (P2) hMSCs, CD133dMSCs, and p75dMSCs cultured in CCM. Note that the freshly isolated stem/progenitor cells are more closely related to each other than to the derived P2 transit-amplifying progenitor cells. The overall transcriptional profiles of the CD133dMSC and p75dMSC subpopulations are more similar to each other than to the profile for typical hMSCs. FIG. 4 (bottom panel) shows a heat map depicting gene expression. Note 9 major patterns with patterns 4, 6, and 9 in particular demonstrating differentially-upregulated genes for hMSCs, p75dMSCs, and CD133dMSCs, respectively. The numbers of transcripts contained in each pattern are shown below with the numbers of uniquely expressed genes shown in parentheses.

FIGS. 5A and 5B show the results of ELISA analysis for selected growth factors/cytokines secreted by hMSCs, CD133dMSCs, and p75dMSCs under normoxic and hypoxic conditions (1% oxygen). Secretion levels for interleukin 6 (IL6), adrenomedullin (ADM), stromal-derived factor 1 (SDF-1) (FIG. 5A), placental growth factor (PLGF), vascular endothelial growth factor (VEGF), hepatocyte growth factor (HGF), and Dickkopf protein 1 (Dkk1) (FIG. 5B) are shown for epitope-sorted MSCs (MACS) or those isolated by simple plastic adherence (hMSCs). All cells were expanded in CCM to the desired confluency, washed twice with PBS, and then incubated in serum free medium for 48 hrs under either normoxic or hypoxic conditions to collect CdM for ELISAs. Data from 3 individual donors for each cell type (P5) are shown at 50% (lower case letters) and 90% (upper case letters) confluencies. Cells obtained by the same isolation method are indicated by horizontal brackets. ELISA data were normalized for cell number. Non-normalized ELISA data in the form of concentration (w/v) are available.

FIGS. 6A and 6B show levels of growth factor/cytokine secretion by hMSCs, CD133dMSCs, and p75dMSCs under normoxic and hypoxic conditions. FIG. 6A shows levels of selected secreted proteins/peptides for cells grown to 50% confluence. FIG. 6B shows levels of selected secreted proteins/peptides for cells grown to 90% confluence. ELISA data were normalized for cell number. Statistical significance values are derived from repeated measures ANOVA.

FIGS. 7A, 7B, and 7C show protection against cerebral ischemia by CD133dMSCs or CD133dMSC conditioned medium (CdM). FIGS. 7A and 7B show tissue sections of murine brain. FIG. 7A shows representative 2,3,5-triphenyltetrazolium chloride (TTC) stains to indicate viable cortical tissue in a sham-operated animal and 1 day or 3 days after middle cerebral artery ligation (MCAL). In sham surgery, the needle is passed under the middle cerebral artery, but the suture is not tied. FIG. 7B shows representative cresyl violet stains of brain sections from immunodeficient mice that underwent middle cerebral artery ligation surgery and treatment 24 hours later with PBS. 2 million human CD133dMSCs, or 40×CD133dMSC CdM. PBS vehicle, CD133dMSCs or CD133dMSC CdM was injected into the left ventricle lumen (intracardiac, 100 ul). Animals were euthanized 48 hrs following treatment for analysis. FIG. 7C shows the quantification of the cortical infarct volumes for PBS-treated (n=5), CD133dMSC-treated (n=6) and CD133dMSC CdM-treated (n=5) mice. *=p≦0.05 compared with PBS, **=p≦0.01 compared with PBS, †=p≦0.05 compared with CD133dMSC administration.

FIGS. 8A and 8B are micrographs showing adult rat cardiac stem/progenitor cells (FIG. 8A) and adult human non-hematopoietic bone marrow stem/progenitor cells (FIG. 8B). FIG. 8A includes four panels, including phase contrast images of cultured cardiac stem cells (CSCs) and cardiac progenitor cells (CPCs) (magnification×200). Cardiac stem cells cultured in modified neural stem cell medium (mNSCM) without serum form spheroids (left). With the addition of 2% FBS to mNSCM (growth medium), cardiac stem cells adhere and can be cultured as cardiac progenitor cells in monolayers (right). FIG. 8B includes four panels, including phase contrast images of MSCs (upper left) and p75MSCs (upper right) (magnification×100). Differentiation of p75MSCs into osteogenic cells that stained with Alizarin red S (lower left) and adipogenic cells that stained with Oil red O (lower right).

FIGS. 9A-9E show the results of CPC proliferation assays. FIG. 9A provides a graph (left panel) showing time course changes in the numbers of CPCs treated with CdM or SFM (left). Data are mean±SEM, n=3 to 7, CdM was assayed from 2 different donors for each cell type. The control cell number (48,896 cells) was regarded as 100%. *, P<0.0001 vs baseline; **, P<0.01 vs baseline; †, P<0.0001 vs SFM. FIG. 9A (right panel) shows phase contrast images of CPCs treated with CdM from MSCs, p75MSCs, or fibroblasts or SFM for 8 days (magnification×100). FIG. 9B is a graph showing time course changes in the numbers of CPCs treated with SFM supplemented with various growth factors (EGF, bFGF, and LIF; 10 ng/ml) in the absence of insulin-transferrin-selenite. Data are mean±SEM, n=3. The control cell number (64,026 cells) was regarded as 100%. *, P<0.01 SFM and SFM+EGF+FGF vs baseline; **, P<0.001 SFM, SFM+EGF+FGF, and SFM+LIF+EGF+FGF vs baseline. Data for growth in 1× CdM from one MSC donor is shown for reference. FIG. 9C is a graph showing that CdM does not support the growth of adult rat cardiac fibroblasts. Data are mean±SEM, n=3. The control cell number (34,606 cells) was regarded as 100%. *, P<0.0001 vs baseline. FIG. 9D is a graph showing the quantification of BrdU-positive CPCs. Data are mean±SEM, n=3. CdM from 2 different donors were assayed for each cell type. *, P<0.05 vs SFM; **, P<0.01 vs SFM. E, Immunoblot for Ki67 in CPCs (molecular weight, 359 kDa). CdM, conditioned medium. SFM, fresh serum-free medium. GM, growth medium (mNSCM supplemented with 2% FBS).

FIG. 10 shows the dose-dependent effect of 10× concentrated CdM on CPC proliferation. Data shown are mean±SEM, n=3. CdM from 2 different donors were assayed for each cell type. CPC growth in 1× CdM from one MSC donor and one p75MSC donor is shown for reference. The control cell number (60,191 cells) was regarded as 100%. *, P<0.0001 vs baseline; **, P<0.0001 vs day 4; †, P<0.05 vs day 8. CdM, conditioned medium.

FIG. 11A-11D shows results of STAT3 activation in CPCs treated with CdM. FIG. 11A (left panel) is an immunoblot showing for phospho-STAT3 and total-STAT3 in CPCs (molecular weight, 86 kDa) The bottom level shows actin levels as a loading control. PC, positive control (HeLa cells treated with interferon-alpha). FIG. 11A (right panel) is a graph showing the quantification of STAT3 phosphorylation. The corrected values in SFM at day 1 and 2 were designated as 1, n=3. *, P<0.05 vs SFM. FIG. 11B provides four micrographs showing immunofluorescence for phospho-STAT3 and total-STAT3 in CPCs (magnification×400). Phospho-STAT3 localizes to CPC nuclei. Blue indicates DAPI nuclear staining. FIG. 11C is a graph that shows the inhibitory effect of AG490 on CPC growth and survival induced by CdM. CPCs were incubated in CdM with or without AG490 10 μM for 48 hours. Data are mean±SEM, n=3 to 6. The control cell numbers (121,863 cells in MSC CdM, 115,342 cells in p75MSC CdM, and 118,682 cells in fibro CdM) were regarded as 100%. *, P<0.0001 vs control. FIG. 11D includes three graphs showing the inhibitory effect of AG490 on CPCs incubated in CdM, SFM and GM for 48 hours. Data are mean±SEM, n=3 to 6. The control cell numbers (121,863 cells in CdM, 99,965 cells in SFM, and 164,614 cells in GM) were regarded as 100%. *, P<0.0001 vs control; **, P<0.01 vs AG; †, P<0.05 vs LY. Con; control, DMSO. AG; AG490 10 μM, Jak2/STAT3 pathway inhibitor. LY; LY294002 10 μM, inhibitor of PI3K/Akt pathway. A+L; AG490 10 μM+LY294002 10 μM. CdM, conditioned medium. CdM, conditioned medium. SFM, fresh serum-free medium. GM, growth medium (mNSCM with 2% FBS).

FIG. 12 is a graph showing that the specific inhibition of STAT3 phosphorylation (Tyr⁷⁰⁵) prevents CPC growth in MSC CdM. ††, P<0.01 for CdM vs. baseline (Day 0); ***, P<0.001 for Stattic vs. CdM.

FIGS. 13A and 13B show the differentiation of CPCs expanded in CdM. FIG. 13A provide a series of micrographs showing immunofluorescent staining for α-SA, α-sarcomeric actin; SMA, α-smooth muscle actin; and vWF, von Willebrand factor (magnification×400). FIG. 13A (left panels, baseline) show the CPCs in growth medium 3 days after plating, and the right panels show CPCs expanded in CdM for 4 days. FIG. 13B provides two graphs showing the quantification of % positive cells for α-SA, SMA, and vWF. Data are mean±SEM, n=3. CdM, conditioned medium.

FIGS. 14A-14D show the protective effect of CdM on CPCs exposed to chronic hypoxia (1% O₂ for 48 hrs). FIG. 14A shows phase contrast images of CPCs treated with SFM (left) or CdM from p75MSCs (right) (magnification×100). FIG. 14B is a graph showing the quantification of cell numbers in GM, SFM and CdM. Data are mean±SEM, n=3, CdM from 2 different donors was assayed for each cell type. The control cell number (139,616 cells) was regarded as 100%. *, P<0.05 vs SFM; **, P<0.01 vs SFM. C, Jak2/STAT3 inhibition blocks protection against hypoxia conferred by CdM. Data are mean±SEM, n=3. The control cell numbers (56,559 cells in MSC CdM, 92,120 cells in p75MSC CdM, and 74,511 cells in fibro CdM) were regarded as 100%. *, P<0.0001 vs CdM. AG; AG490 10 μM, Jak2/STAT3 pathway inhibitor. CdM, conditioned medium. SFM, fresh serum-free medium. GM, growth medium (mNSCM with 2% FBS). D, Specific inhibition of STAT3 phosphorylation (Tyr⁷⁰⁵) prevents CPC protection by MSC CdM during chronic hypoxia exposure. †, P<0.05 for CdM vs. SFM; **, P<0.001 for Stattic vs. CdM; ***, P<0.0001 for Stattic vs. CdM.

FIGS. 15A and 15B are graphs showing the results of intra-arterial administration of concentrated conditioned medium from CD133dMSCs and p75dMSCs on cardiac function 1 week after myocardial infarction (MI). FIG. 15A shows that P75 CdM and CD133 CdM significantly improve wall motion (thickening) after myocardial infarction. Echocardiography score was determined with a 13 segment model similar to the American Society of Echocardiography's 16 segment model. The best possible score is a 13 and the worst possible score is a 39. Echocardiography was performed using a VisualSonics Vevo 770 system. SFM vs. p75 CdM, p≦0.05; SFM vs. CD133 CdM, p≦0.01. FIG. 15 shows that P75 CdM and CD133 CdM significantly increase (preserve) the percent of fractional shortening after myocardial infarction (MI). SFM vs. p75 CdM, p≦0.01; SFM vs. CD133 CdM, p≦0.05. For all of the data, SFM, n=8; p75 CdM, n=6; CD133 CdM, n=4.

FIGS. 16A and 16B are graphs showing that intra-arterial administration of concentrated conditioned medium from CD133dMSCs and p75dMSCs leads to improved cardiac function 1 week after myocardial infarction (MI). A) P75 CdM significantly increases (preserves) anterior wall thickness in diastole after MI. Echocardiography was performed using a VisualSonics Vevo 770 system. SFM vs. p75 CdM, p≦0.05; SFM vs. CD133 CdM, NS. B) P75 CdM and CD133 CdM significantly increase (preserve) anterior wall thickness in systole after MI. SFM vs. p75 CdM, p≦0.05; SFM vs. CD133 CdM, p≦0.05. For all of the data, SFM, n=8; p75 CdM, n=6; CD133 CdM, n=4.

FIGS. 17A and 17B are graphs showing that intra-arterial administration of concentrated conditioned medium from CD133dMSCs and p75dMSCs leads to improved cardiac function 1 week after myocardial infarction (MI). FIG. 17A shows no significant difference in end diastolic diameter of the left ventricle with or without p75 CdM or CD133 CdM treatment after MI. Echocardiography was performed using a VisualSonics Vevo 770 system. FIG. 17B shows that P75 CdM and CD133 CdM significantly decrease the end systolic diameter of the left ventricle after MI. SFM vs. p75 CdM, p≦0.05; SFM vs. CD133 CdM, p≦0.01. For all of the data, SFM, n=8; p75 CdM, n=6; CD133 CdM, n=4.

FIGS. 18A and 18B show that CD133dMSC conditioned medium (CdM) protects against cellular damage due to cerebral ischemia. FIG. 18A provides representative cresyl violet stains of brain sections from immunodeficient mice that underwent permanent middle cerebral artery ligation (MCAL) surgery and received treatment 24 hours later with PBS, 2 million human CD133dMSCs, or concentrated CdM from p75dMSCs, CD133dMSCs, or typical hMSCs (MSC). The PBS vehicle, CD133dMSCs or CdM from the different cell types was injected into the left ventricle of the heart (intra-arterial, 100 μl). Animals were euthanized 48 hrs following treatment for analysis (3 d after pMCAL). Scale bars=1 mm. FIG. 18B is a graph showing quantification of the cortical infarct volumes for PBS-treated (n=5), p75dMSC CdM-treated (n=5), CD133dMSC-treated (n=6), CD133dMSC CdM-treated (n=5), and MSC CdM-treated mice (n=5). A single asterisk (*) signifies p<0.05 when compared with PBS. A double asterisk (**) denotes p<0.01 compared with PBS. Statistics were determined by ANOVA with Bonferroni post-hoc testing. To calculate infarct volumes a 20 micron section was quantified every 200 microns through the zone of infarction and multiplied by 10 to determine the total infarct volume (NIH Image J).

FIG. 19 is a graph quantitating improved motor function in CD133dMSC CdM-treated mice at 1 month after stroke.

FIGS. 20A-20C show that CD133dMSCs significantly increased expression SDF-1 mRNA when injected adjacent to the injured cerebral cortex after MCAL. FIG. 20A is a micrograph showing GFP fluorescence from CD133dMSCs 48 hrs after injection into peri-infarct area (red autofluorescence shows stroke core). FIG. 20B is a graph quantitating results of human-specific real time PCR to detect mRNAs of GAPDH and secreted proteins. FIG. 20C is a graph showing relative mRNA levels in MCAL brains compared with sham brains (no ligation) 48 hrs after being injected with GFP-CD133dMSCs. For both sham brains and MCAL brains, the mRNA level of the human growth factor/cytokine mRNA was normalized to the level of human GAPDH mRNA in the sample. For each mRNA, the GAPDH normalized level in sham is set to 1. SDF1 mRNA levels increased by 79 fold in MCAL brains versus sham brains (sham, n=10; MCAL n=12; ** P<0.01 compared with sham).

FIGS. 21A and 21B show the transduction of CD133dMSCs with puromycin-selectab lentivectors expressing GFP, scrambled (non-specific) shRNA or sequence-specific shRNA (against SDF-1). FIG. 21A shows results of flow cytometry analysis (FACS) of control cells (no label) and those transduced with GFP vector and selected by puromycin to determine cell purity after selection. FIG. 21B is a graph quantitating SDF1 secretion as assayed by ELISA of conditioned medium from control CD133dMSCs (untransduced, CD133 Con), those transduced by lentivector with scrambled shRNA (shRNA Scram), and those transduced with 2 different lentivectors with SDF1 shRNAs (shRNA1 SDF-1, shRNA2 SDF-1). Medium conditioned for 48 hrs in a 6 well plate by equal cell numbers was assayed in each case.

FIGS. 22A-22C show that secreted SDF-1 from CD133dMSCs protects mouse neural progenitor cells (mNPCs) under hypoxic/ischemic conditions. FIG. 22A is a micrograph showing the isolation (neural spheres) and differentiation of postnatal day 4 (D4) mNPCs from GFP mice. Beta III tubulin staining indicates neuronal differentiation and GFAP indicates astrocytic differentiation after 1 week in the relevant differentiation mediums. FIG. 22B is a graph showing that CD133dMSC CdM provides significant protection of mNPCs during growth factor withdrawal. Surviving NPC numbers were normalized to those that received CD133dMSC CdM prior to hypoxia exposure. Interestingly, CdM from the p75dMSC subpopulation did not provide protection. CD133, N=3 donors; p′75, N=3 donors; hMSC, N=2 donors. FIG. 22C a graph showing that SDF-1 is one of the factors contained in CD133dMSC CdM that provides protection of mNPCs during exposure to hypoxia/ischemia. CdM from control CD133dMSCs (CD133 Con) and CdM from CD133dMSCs transduced with a scrambled shRNA lentivector (shRNA Scram) both protected against hypoxia/ischemia exposure (P ≦0.01 compared with SFM). Lentiviral transduction of CD133dMSCs with a SDF-1 specific shRNA (shRNA SDF-1 significantly reduced the level of mNPC protection conferred by CdM (P ≦0.01 compared with shRNA Scram CdM). Surviving NPC numbers were normalized to those that received new mNSCM growth media prior to hypoxia exposure. All assays were performed in triplicate.

### DETAILED DESCRIPTION OF THE INVENTION

The invention features compositions comprising mesenchymal stem cells or multipotent stromal cells, agents secreted by such cells in culture, and methods featuring such cells for the repair or regeneration of a damaged tissue or organ.

The present invention is based, at least in part, on the discovery that media isolated from bone marrow mesenchymal stem cells or multipotent stromal cells (MSCs) provided neuroprotection in vivo following cerebral ischemia. Surprisingly, these cells secreted factors that reduced cell death, negatively regulated inflammatory responses, and promoted the healing of injured tissues. As reported in more detail below, human multipotent stromal cells (hMSCs) were compared with multipotent non-hematopoietic progenitor cell subpopulations that were isolated by magnetic-activated cell sorting against the CD133 epitope (CD133-derived multipotent stromal cells, CD133dMSCs) or CD271 (p75LNGFR, p75-derived multipotent stromal cells). Microarray assays of expressed genes demonstrated that the three transit-amplifying progenitor cell populations were distinct from one another. The secretion levels of selected growth factors and cytokines at different cell densities were analyzed under normoxic or hypoxic conditions (1% oxygen). The human multipotent stromal cells, CD133-derived multipotent stromal cells, and p75-derived multipotent stromal cells secreted significantly different levels of IL6, VEGF, PLGF, SDF1, HGF, DKK1, and adrenomedullin when cultured in normoxia or hypoxia for 48 hours. Many reports have demonstrated that multipotent stromal cells, and secreted factors from multipotent stromal cells, have great therapeutic potential. To determine whether epitope-isolated subpopulations of multipotent stromal cells also possessed such potential, intracardiac (arterial) administrations of CD133-derived multipotent stromal cells or concentrated conditioned medium from CD133-derived multipotent stromal cells was tested. Surprisingly, this administration provided neuroprotection and dramatically reduced cortical infarct volumes in mice following cerebral ischemia.

The present invention is also based in part on the discovery that serum-free conditioned medium enhanced the growth, survival, and/or proliferation of cardiac progenitor cells. Condioned media (CDM) was collected from human MSCs that were isolated by plastic adherence (MSCs) and by magnetic sorting against the p75 nerve-growth factor receptor (p75MSCs). Condioned media obtained from such cells supported the proliferation of cardiac progenitor cells isolated from adult rat heart. Compared with baseline (100%), cardiac progenitor cells incubated in fresh serum-free medium decreased (45.1%). In contrast, cardiac progenitor cells incubated in condioned media increased (MSCs, 143.4%; p75MSCs, 147.5%; p<0.001 vs serum-free medium at day 8). There was a concentration-dependent increase in cardiac progenitor cell number when the Cardiac progenitor cells were incubated in 10×-concentrated condioned media. Growth of cardiac progenitor cells in condioned media led to phosphorylation and nuclear localization of signal transducer and activator of transcription 3 (STAT3). AG490, a Janus kinase 2 (Jak2)/STAT3 pathway inhibitor, and Stattic, a specific STAT3 inhibitor, blocked the CdM-induced proliferation of cardiac progenitor cell. The condioned media-expanded cardiac progenitor cell remained multipotent and differentiated into several cardiac cell types. Condioned media from MSCs increased the survival of cardiac progenitor cells exposed to hypoxia (1% oxygen for 48 hrs) compared with serum-free medium (≈1.6 fold increase, P<0.05). The protective factors in MSC condioned media also signaled through the Jak2/STAT3 pathway. Based on these results, it is likely that factors secreted by MSCs activate STAT3 in cardiac progenitor cell, promote their proliferation, and protect them from hypoxic injury. The beneficial effects of MSCs in vivo may be mediated in part by the action of their secreted factors on cardiac progenitor cells. Incubation of cardiac progenitor cells in conditioned medium from MSCs or p75MSCs led to phosphorylation of signal transducer and activator of transcription 3 (STAT3), thereby increasing the growth and survival of cardiac progenitor cells.

The beneficial effects of conditioned media on cardiac progenitor cells were not limited to cells in culture, but also showed a therapeutic effect when administered in vivo following myocardial infarction. Mice that received conditioned media following myocardial infarction showed a marked increase in cardiac function relative to untreated control mice.

In sum, the results reported herein indicate that conditioned media from non-hematopoietic multipotent stromal cells may be used to support the repair or regeneration of a variety of organs by reducing cell death, negatively regulating inflammatory responses, and promoting the healing of injured tissues. Such beneficial effects are likely related to an increase in the growth, proliferation, or survival of specific populations of progenitor cells or stem cells capable of repairing the damaged tissue or organ.

### Stem Cells

Adult mammalian bone marrow contains hematopoietic stem cells (HSCs) and progenitor cells that produce all of the major blood cell lineages. The field of HSC biology has benefited greatly from functional reconstitution assays in mice in which fractionated cell subsets can be transplanted into irradiated recipients to determine cell lineage relationships. In this manner, characterization of cell surface epitopes and transplantation of HSCs and upstream progenitors identified the two functionally distinct branches of the hematopoietic system that derive from common myeloid progenitor cells and common lymphoid progenitor cells.

Adult bone marrow also contains non-hematopoietic stem and progenitor cells that contribute to the hematopoietic microenvironment and provide circulating reparative cells for non-hematopoietic tissues. An elegant recent study demonstrated that CD146-positive cells isolated from human bone marrow contained non-hematopoietic stem-like cells that could be expanded and serially transplanted to transfer ectopic hematopoietic microenvironments (Sacchetti et al. Cell. 2007; 131: 324-336). In its native environment, the non-hematopoietic bone marrow stem cell is likely to produce the progenitor cells commonly described as mesenchymal stem cells or multipotent stromal cells (MSCs), which in part contribute structurally to the endosteal and sinusoidal compartments of the marrow that comprise HSC niches. MSCs function in regulating HSC proliferation, differentiation, and quiescence in vivo by signaling via the “stem cell niche synapse” through which growth factors, cytokines, and immunomodulatory factors are exchanged. MSCs are adherent in culture, are identified by their ability to differentiate into stromal cells, osteoblasts, adipocytes and chondrocytes (Prockop, Science. 1997; 276: 71-74; Pittenger et al., Science. 1999; 284:143-147), and support the ex vivo maintenance of HSCs through their secreted factors and production of extracellular matrix components (Dexter et al., Prog. Clin. Biol. Res. 1984; 148: 13-33; Gualtieri et al., Blood. 1984; 64: 516-525; Ueno et al., Nature Immunol. 2003; 4: 457-463). In addition to regulating hematopoiesis, MSCs and related cells may also enter the circulation and serve as a “continuous reservoir” of replacement cells and/or reparative cells for non-hematopoietic tissues. Despite several decades of MSC research, in contrast to HSC biology where different progenitor cell lineages are known by distinct cell surface epitopes and functions, the progeny of the non-hematopoietic bone marrow stem cell remain relatively poorly defined. For example, it is still not clear whether or not there are single or multiple lineages of multipotent non-hematopoietic progenitors as was previously delineated for the hematopoietic system.

MSCs from bone marrow and other tissues have received increasing attention as expandable cells that can be used for cell and gene therapy (Prockop et al., Proc. Natl. Acad. Sci. USA. 2003; 100: 11917-11923). They have been demonstrated to provide functional benefits in a wide variety of animal models for tissue injury and disease such as myocardial infarction (Mangi et al., Nat Med. 2003; 9:1195-1201; Iso et al., Biochem. Biophys. Res. Commun. 2007; 354: 700-706), hind limb ischemia (Kinnaird et al., Circulation. 2004; 109: 1543-1549), and stroke (Chen et al., Stroke. 2001; 32:1005-1011). Positive results have also been reported in patients that received MSCs (Horwitz et al., Nat Med. 1999; 5:309-13; Le Blanc et al. Lancet. 2008; 371:1579-1586; Koç et al., J Clin Oncol. 2000; 18:307-316). However, low long-term MSC engraftment numbers reported in both animals and patients suggests that cell replacement is not the predominant mechanism responsible for the benefits conferred by MSC administrations. Rather, it has been suggested that MSCs are secreting “factories” that rescue cells, repair tissues, and provide improved functional outcomes by virtue of their secretion of a multitude of growth factors, cytokines, and immunomodulatory molecules, but data supporting this suggestion has been lacking.

MSCs are commonly isolated from bone marrow aspirates by density gradient centrifugation to obtain mononuclear cells and then by simple adherence to tissue culture plastic and rapid growth in supportive mediums. These conditions, however, do not select for any particular progenitor cell population and it is not clear that the MSCs isolated by different laboratories actually represent the same cells. The lack of standardization likely leads to differing results reported by some investigators that administer MSCs to treat similar animal models of tissue injury and disease. It is generally assumed that the transit-amplifying progenitors that expand from adherent bone marrow cultures and that possess a defined set of cell surface epitopes are functionally equivalent.

In the interests of developing safe and effective cell therapies with predictable effects in vivo, non-hematopoietic progenitor cells were isolated directly from human bone marrow mononuclear cells by magnetic-activated cell sorting (MACS) against two different cell surface epitopes (CD133, Prominin 1) (Tondreau et al., Stem Cells. 2005; 23:1105-1112), and bone marrow (CD271, p75-low affinity nerve growth factor receptor, p75LNGFR) (Quirici et al., Exp Hematol. 2002; 30:783-791). While similar to typically-isolated human MSCs (hMSCs) in several ways, the CD133-derived MSCs (CD133dMSCs) and the p75LNGFR-derived MSCs (p75dMSCs) differed from typical hMSCs and from each other in terms of their secreted growth factors and cytokines. In addition, the hMSCs, CD133dMSCs, and p75dMSCs had different secretion responses when exposed to hypoxic environments, indicating that the non-hematopoietic bone marrow stem cell likely produce different progenitors that reside in different marrow environments. Based on these results it is likely that MSC subpopulations from the bone marrow or other tissues may be reproducibly isolated and exploited in tailor made cell-based therapies for tissue injury and disease on the basis of differential growth factor and cytokine secretion.

### Individualized Therapies

In one approach, the invention provides cellular compositions derived from a subject having or at risk of developing a disease or disorder characterized by a deficiency in cell number, such as an ischemic injury. Such cellular compositions comprise MSC subpopulations from the bone marrow or other tissues that are isolated from the subject prior to the injury. Such cells are then cultured in vitro to obtain culture media comprising agents that support tissue repair or regeneration. Preferably, the culture media is purified to yield a therapeutic composition comprising biologically active agents in a pharmaceutically acceptable excipients. If desired, such compositions further comprise cryoprotective agents that enhance the biological activity of the agents when frozen for a period of months or years and then subsequently thawed. Alternatively, cells derived from the subject are stored frozen, thawed, and cultured in vitro to obtain a therapeutic composition comprising agents that support tissue repair or regeneration. Advantageously, the invention provides for reproducible individualized cell-based therapies for tissue injury and disease and therapeutic compositions comprising agents having biological activity (e.g., agents that reduce cell death, negatively regulate inflammation, promote an increase in cell growth, proliferation, or survival). Such therapeutic compositions likely comprise a unique combination of growth factors and cytokines secreted by cells isolated and cultured according to the methods of the invention. Such methods provide for therapeutic compositions having combinations of factors that are unexpectedly potent in preventing or ameliorating the effects of ischemic injury.

Accordingly, the present invention provides methods of treating disease and/or disorders characterized by tissue damage, undesirable cell death, or a cellular deficiency, or symptoms thereof which comprise administering a therapeutically effective amount of a pharmaceutical composition comprising a cell or composition delineated herein to a subject (e.g., a mammal such as a human). Thus, one embodiment is a method of treating a subject suffering from or susceptible to tissue damage relating to an ischemic disease or disorder or symptom thereof. The method includes the step of administering to the mammal a therapeutic amount of an amount of a compound herein sufficient to treat the tissue damage, ischemic disease or disorder or symptom thereof, under conditions such that the disease or disorder is treated.

The methods herein include administering to the subject (including a subject identified as in need of such treatment) an effective amount of a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a composition delineated herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof (e.g., susceptible to ischemic injury, such as heart attack or stroke). Determination of those subjects “at risk” can be made by any objective or subjective determination by a diagnostic test or opinion of a subject or health care provider (e.g., genetic test, enzyme or protein marker, Marker (as defined herein), family history, and the like). The compositions herein may be also used in the treatment of any other disorders in which tissue damage may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with tissue damage, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Isolation of Cells

While the results reported herein provide specific examples of the isolation of mesenchymal stem cells or multipotent stromal cells from bone marrow, the invention is not so limited. The unpurified source of cells for use in the methods of the invention may be any tissue or organ known in the art. In various embodiments, cells of the invention are isolated from adult bone marrow, peripheral blood, or cord blood. Preferably, cells of the invention are non-hematopoietic progenitor cells selected for expression of CD133 or CD271/p75-low affinity nerve growth factor receptor. Various techniques can be employed to separate or enrich for the desired cells. Such methods include a positive selection for cells expressing these markers. Monoclonal antibodies are particularly useful for identifying markers associated with the desired cells. If desired, negative selection methods can be used in conjunction with the methods of the invention to reduce the number of irrelevant cells present in a population of cells selected for CD133 or CD271 expression.

In one approach, magnetic-activated cell sorting (MACS) is used to select for the desired cell type. Other procedures which may be used for selection of cells of interest include, but are not limited to, fluorescence based cell sorting, density gradient centrifugation, flow cytometry, magnetic separation with antibody-coated magnetic beads, cytotoxic agents joined to or used in conjunction with a mAb, including, but not limited to, complement and cytotoxins; and panning with antibody attached to a solid matrix or any other convenient technique. The cells can be selected against dead cells, by employing dyes associated with dead cells such as propidium iodide (PI). Preferably, the cells are collected in a medium comprising fetal calf serum (FCS) or bovine serum albumin (BSA) or any other suitable, preferably sterile, isotonic medium. Selected cells of the invention may be employed in therapeutic or prophylactic methods following isolation or may be grown for a period of time in vitro.

The selected cells may be grown in culture for hours, days, or even weeks during which time their culture medium becomes enriched in biologically active agents that enhance tissue repair or reduce cell death. Media enriched for such biologically active agents is termed “conditioned media.” Biologically active agents present in the conditioned media are useful to enhance tissue repair or to reduce apoptosis. Media and reagents for tissue culture are well known in the art (see, for example, Pollard, J. W. and Walker, J. M. (1997) Basic Cell Culture Protocols, Second Edition, Humana Press, Totowa, N.J.; Freshney, R. I. (2000) Culture of Animal Cells, Fourth Edition, Wiley-Liss, Hoboken, N.J.). Examples of suitable media for incubating mesenchymal stem cells or multipotent stromal cells samples include, but are not limited to, Dulbecco's Modified Eagle Medium (DMEM), RPMI media, Hanks' Balanced Salt Solution (HBSS) phosphate buffered saline (PBS) and other media known in the art. Examples of appropriate media for culturing cells of the invention include, but are not limited to, Dulbecco's Modified Eagle Medium (DMEM), RPMI media. The media may be supplemented with fetal calf serum (FCS) or fetal bovine serum (FBS) as well as antibiotics, growth factors, amino acids, inhibitors or the like, which is well within the general knowledge of the skilled artisan.

### Formulations

In one embodiment, a composition of the invention comprises purified cells, such as mesenchymal stem cells or multipotent stromal cells from bone marrow, in particular non-hematopoietic progenitor cells selected for expression of CD 133 or CD271/p75-low affinity nerve growth factor receptor or their progeny. If desired, such cellular compositions may be administered to a subject for tissue repair or regeneration. In other embodiments, a composition of the invention comprises conditioned media obtained during the culture of such cells that contains biologically active agents secreted by a cell of the invention. The biologically active agents present in the condition media, the cells, or a combination thereof, can be conveniently provided to a subject as sterile liquid preparations, e.g., isotonic aqueous solutions, suspensions, emulsions, dispersions, or viscous compositions, which may be buffered to a selected pH. Cells and agents of the invention may be provided as liquid or viscous formulations. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions are prepared by incorporating cells of the invention or compositions comprising biologically active agents present in the conditioned media isolated from cultures of such cells in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

Compositions comprising a cell of the invention (e.g., mesenchymal stem cells or multipotent stromal cells) will typically comprise a quantity of cells necessary to achieve an optimal therapeutic or prophylactic effect. The quantity of cells to be administered will vary for the subject being treated. In a one embodiment, between 10⁴ to 10⁸, between 10⁵ to 10⁷, or between 10⁶ and 10⁷ genetically mesenchymal stem cells or multipotent stromal cells of the invention are administered to a human subject. In preferred embodiments, at least about 1×10⁷, 2×10⁷, 3×10⁷, 4×10⁷, and 5×10⁷ cells are administered to a human subject.

Compositions comprising biologically active agents present in conditioned media are also administered in an amount required to achieve a therapeutic or prophylactic effect. Such an amount will vary depending on the conditions of the culture. Typically, biologically active agents present in the conditioned media will be purified and subsequently concentrated so that the protein content of the composition is increased by at least about 5-fold, 10-fold or 20-fold over the amount or protein originally present in the media. In other embodiments, the protein content is increased by at least about 25-fold, 30-fold, 40-fold or even by 50-fold.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

### Methods of Delivery

Compositions comprising a cell of the invention (e.g., a non-hematopoietic progenitor cell selected for expression of CD133 or CD271/p75-low affinity nerve growth factor receptor) or a composition comprising biologically active agents present in conditioned media are provided systemically or directly to a site of injury. Modes of administration include intramuscular, intra-cardiac, oral, rectal, topical, intraocular, buccal, intravaginal, intracisternal, intracerebroventricular, intratracheal, nasal, transdermal, within/on implants, e.g., fibers such as collagen, osmotic pumps, or parenteral routes. The term “parenteral” includes subcutaneous; intravenous, intramuscular, intraperitoneal, intragonadal or infusion.

In one approach, cells derived from cultures of the invention are implanted into a host. The transplantation can be autologous, such that the donor of the cells is the recipient of the transplanted cells; or the transplantation can be heterologous, such that the donor of the cells is not the recipient of the transplanted cells. Once transferred into a host, the cells are engrafted, such that they assume the function and architecture of the native host tissue. In particular embodiments, at least 100,000, 250,000, or 500,000 cells is injected. In other embodiments, 750,000, or 1,000,000 cells is injected. In other embodiments, at least about 1×10⁵ cells will be administered, 1×10⁶, 1×10⁷, or even as many as 1×10⁸ to 1×10¹⁰, or more are administered.

Selected cells of the invention comprise a purified population of non-hematopoietic progenitor cells selected for expression of CD133 or CD271/p75-low affinity nerve growth factor receptor. Those skilled in the art can readily determine the percentage of cells in a population using various well-known methods, such as fluorescence activated cell sorting (FACS). Preferable ranges of purity in populations comprising selected cells are about 50 to about 55%, about 55 to about 60%, and about 65 to about 70%. More preferably the purity is at least about 70%, 75%, or 80% pure, more preferably at least about 85%, 90%, or 95% pure. In some embodiments, the population is at least about 95% to about 100% selected cells. Dosages can be readily adjusted by those skilled in the art (e.g., a decrease in purity may require an increase in dosage). The cells can be introduced by injection, catheter, or the like. Compositions of the invention include pharmaceutical compositions comprising biologically active agents present in conditioned media and a pharmaceutically acceptable carrier. Administration can be autologous or heterologous. For example, non-hematopoietic progenitor cells selected for expression of CD133 or CD271/p75-low affinity nerve growth factor receptor can be obtained from one subject, and administered to the same subject or a different, compatible subject. Selected cells of the invention or the biologically active agents present in conditioned media obtained from the culture of such cells an be administered via localized injection, including catheter administration, systemic injection, localized injection, intravenous injection, or parenteral administration. When administering a therapeutic composition of the present invention, it will generally be formulated in a unit dosage injectable form (solution, suspension, emulsion).

If desired, a cell of the invention (e.g., a non-hematopoietic progenitor cell selected for expression of CD133 or CD271/p75-low affinity nerve growth factor receptor or its in vitro-derived progeny) and/or biologically active agents present in conditioned media are incorporated into a polymer scaffold to promote tissue repair, cell survival, proliferation in a tissue in need thereof. Polymer scaffolds can comprise, for example, a porous, non-woven array of fibers. The polymer scaffold can be shaped to maximize surface area, to allow adequate diffusion of nutrients and growth factors to a cell of the invention. Polymer scaffolds can comprise a fibrillar structure. The fibers can be round, scalloped, flattened, star-shaped, solitary or entwined with other fibers. Branching fibers can be used, increasing surface area proportionately to volume.

Unless otherwise specified, the term “polymer” includes polymers and monomers that can be polymerized or adhered to form an integral unit. The polymer can be non-biodegradable or biodegradable, typically via hydrolysis or enzymatic cleavage. The term “biodegradable” refers to materials that are bioresorbable and/or degrade and/or break down by mechanical degradation upon interaction with a physiological environment into components that are metabolizable or excretable, over a period of time from minutes to three years, preferably less than one year, while maintaining the requisite structural integrity. As used in reference to polymers, the term “degrade” refers to cleavage of the polymer chain, such that the molecular weight stays approximately constant at the oligomer level and particles of polymer remain following degradation.

Materials suitable for polymer scaffold fabrication include polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl chloride, polyvinyl flouride, polyvinyl imidazole, chlorosulphonated polyolifins, polyethylene oxide, polyvinyl alcohol, Teflon®, nylon silicon, and shape memory materials, such as poly(styrene-block-butadiene), polynorbornene, hydrogels, metallic alloys, and oligo(ε-caprolactone)diol as switching segment/oligo(p-dioxyanone)diol as physical crosslink. Other suitable polymers can be obtained by reference to The Polymer Handbook, 3rd edition (Wiley, N.Y., 1989).

### Expression of Recombinant Proteins

In another approach, a cell of the invention (e.g., a non-hematopoietic progenitor cell selected for expression of CD133 or CD271/p75-low affinity nerve growth factor receptor) or its in vitro-derived progeny is engineered to express a gene of interest whose expression promotes cell survival, proliferation, differentiation, engraftment of the cell, reduces cell death, or otherwise contributes to tissue repair. For example, expression of a gene of interest in a cell of the invention may promote the repair of a tissue or organ having a deficiency in cell number or excess cell death due to ischemic injury, such as stroke or myocardial infarction. Alternatively, cells of the invention may express a component of the extracellular matrix (ECM), such as Wnt/Beta catenin pathway (wild-type and stable mutant beta catenin), ramp up secretion signal, increased Notch pathway (Notch intercellular domain). In one embodiment, such cells are selected using any type of affinity based selection. In another embodiment, cell express an ECM component encoded by a lentivector that is doxycycline inducible.

Virtually any vector or delivery system known in the art may be used to modify a cell of the invention (e.g., bone marrow derived MSC or progenitor thereof). Preferably, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997).

Non-viral approaches can be employed for the expression of a protein in cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346).

The gene of interest may be constitutively expressed or its expression may be regulated by an inducible promoter or other control mechanism where conditions necessitate highly controlled regulation or timing of the expression of a protein, enzyme, or other cell product. Such cells, when transplanted into a subject, produce high levels of the protein to confer a therapeutic benefit. Insertion of one or more pre-selected DNA sequences can be accomplished by homologous recombination or by viral integration into the host cell genome. The desired gene sequence can also be incorporated into the cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

### Screening Assays

The invention provides methods for identifying biologically active agents present in the conditioned media of a cell of the invention (e.g., a non-hematopoietic progenitor cell selected for expression of CD133 or CD271/p75-low affinity nerve growth factor receptor). Such agents include proteins, peptides, polynucleotides, small molecules or other agents that enhance tissue repair. Agents thus identified can be used to enhance tissue repair by modulating, for example, the proliferation, survival, or differentiation of cells of the tissue of interest. In one embodiment, agents identified according to a method of the invention reduce apoptosis.

The test agents of the present invention can be obtained singly or using any of the numerous approaches. Such methods will typically involve contacting a population of cells at risk of cell death with a test agent isolated from conditioned media and measuring an increase in survival or a reduction in cell death as a result of the contact. Comparison to an untreated control can be concurrently assessed. Where an increase in the number of surviving cells or a reduction in cell death is detected relative to the control, the test agent is determined to have the desired activity.

Fractionation of the conditioned media will be necessary to isolate chemical constituents having a desired biological activity. Thus, the goal of the extraction, fractionation, and purification process is the careful characterization and identification of a chemical entity within the conditioned media having the desired biological activity. Methods of fractionation and purification of such heterogenous extracts are known in the art. If desired, peptides, polynucleic acids, or small compounds shown to be useful agents for enhancing tissue repair are chemically modified according to methods known in the art. Such agents may be characterized for biological activity in using methods known in the art, including animal models of tissue injury and disease such as myocardial infarction, hind limb ischemia, and stroke.

### Methods for Evaluating Therapeutic Efficacy

In one approach, the efficacy of the treatment is evaluated by measuring, for example, the biological function of the treated organ (e.g., bladder, bone, brain, breast, cartilage, esophagus, fallopian tube, heart, pancreas, intestines, gallbladder, kidney, liver, lung, nervous tissue, ovaries, prostate, skeletal muscle, skin, spinal cord, spleen, stomach, testes, thymus, thyroid, trachea, ureter, urethra, urogenital tract, and uterus). Such methods are standard in the art and are described, for example, in the Textbook of Medical Physiology, Tenth edition, (Guyton et al., W.B. Saunders Co., 2000). Preferably, a method of the present invention, increases the biological function of a tissue or organ by at least 5%, 10%, 20%, 40%, 50%, 60%, 70%, 80%, 90%, 100%, 150%, 200%, or even by as much as 300%, 400%, or 500%.

In another approach, the therapeutic efficacy of the methods of the invention is assayed by measuring an increase in cell number in the treated or transplanted tissue or organ as compared to a corresponding control tissue or organ (e.g., a tissue or organ that did not receive treatment). Preferably, cell number in a tissue or organ is increased by at least 5%, 10%, 20%, 40%, 60%, 80%, 100%, 150%, or 200% relative to a corresponding tissue or organ. Methods for assaying cell proliferation are known to the skilled artisan and are described, for example, in Bonifacino et al., (Current Protocols in Cell Biology Loose-leaf, John Wiley and Sons, Inc., San Francisco, Calif.). For example, assays for cell proliferation may involve the measurement of DNA synthesis during cell replication. In one embodiment, DNA synthesis is detected using labeled DNA precursors, such as [³H]-Thymidine or 5-bromo-2*-deoxyuridine [BrdU], which are added to cells (or animals) and then the incorporation of these precursors into genomic DNA during the S phase of the cell cycle (replication) is detected (Ruefli-Brasse et al., Science 302(5650):1581-4, 2003; Gu et al., Science 302 (5644):445-9, 2003).

In another approach, efficacy is measured by detecting an increase in the number of viable cells present in a tissue or organ relative to the number present in an untreated control tissue or organ, or the number present prior to treatment. Assays for measuring cell viability are known in the art, and are described, for example, by Crouch et al. (J. Immunol. Meth. 160, 81-8); Kangas et al. (Med. Biol. 62, 338-43, 1984); Lundin et al., (Meth. Enzymol. 133, 27-42, 1986); Petty et al. (Comparison of J. Biolum. Chemilum. 10, 29-34, 1995); and Cree et al. (AntiCancer Drugs 6: 398-404, 1995). Cell viability can be assayed using a variety of methods, including MTT (3-(4,5-dimethylthiazolyl)-2,5-diphenyltetrazolium bromide) (Barltrop, Bioorg. & Med. Chem. Lett. 1: 611, 1991; Cory et al., Cancer Comm. 3, 207-12, 1991; Paull J. Heterocyclic Chem. 25, 911, 1988). Assays for cell viability are also available commercially. These assays include but are not limited to CELLTITER-GLO® Luminescent Cell Viability Assay (Promega), which uses luciferase technology to detect ATP and quantify the health or number of cells in culture, and the CellTiter-Glo® Luminescent Cell Viability Assay, which is a lactate dehyrodgenase (LDH) cytotoxicity assay (Promega).

Alternatively, or in addition, therapeutic efficacy is assessed by measuring a reduction in apoptosis. Apoptotic cells are characterized by characteristic morphological changes, including chromatin condensation, cell shrinkage and membrane blebbing, which can be clearly observed using light microscopy. The biochemical features of apoptosis include DNA fragmentation, protein cleavage at specific locations, increased mitochondrial membrane permeability, and the appearance of phosphatidylserine on the cell membrane surface. Assays for apoptosis are known in the art. Exemplary assays include TUNEL (Terminal deoxynucleotidyl Transferase Biotin-dUTP Nick End Labeling) assays, caspase activity (specifically caspase-3) assays, and assays for fas-ligand and annexin V. Commercially available products for detecting apoptosis include, for example, Apo-ONE® Homogeneous Caspase-3/7 Assay, FragEL TUNEL kit (ONCOGENE RESEARCH PRODUCTS, San Diego, Calif.), the ApoBrdU DNA Fragmentation Assay (BIOVISION, Mountain View, Calif.), and the Quick Apoptotic DNA Ladder Detection Kit (BIOVISION, Mountain View, Calif.).

### Kits

Compositions comprising a cell of the invention (e.g., a non-hematopoietic progenitor cell selected for expression of CD133 or CD271/p75-low affinity nerve growth factor receptor) or a composition comprising biologically active agents present in conditioned media of such cells is supplied along with additional reagents in a kit. The kits can include instructions for the treatment regime, reagents, equipment (test tubes, reaction vessels, needles, syringes, etc.) and standards for calibrating or conducting the treatment. The instructions provided in a kit according to the invention may be directed to suitable operational parameters in the form of a label or a separate insert. Optionally, the kit may further comprise a standard or control information so that the test sample can be compared with the control information standard to determine if whether a consistent result is achieved.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

Current methods for tissue repair, particularly current therapeutic methods for treating or preventing ischemic tissue damage are inadequate. Accordingly, in the interests of developing safe and effective cell therapies with predictable effects in vivo, non-hematopoietic progenitor cells were isolated directly from human bone marrow mononuclear cells by magnetic-activated cell sorting (MACS) against CD133 (Prominin 1) and CD271, also termed the p75-low affinity nerve growth factor receptor, p75LNGFR).

### Example 1

### P2 CD133- and p75-Derived MSCs Expressed High Levels of CD146, a Marker for Human Non-Hematopoietic Bone Marrow Stem Cells

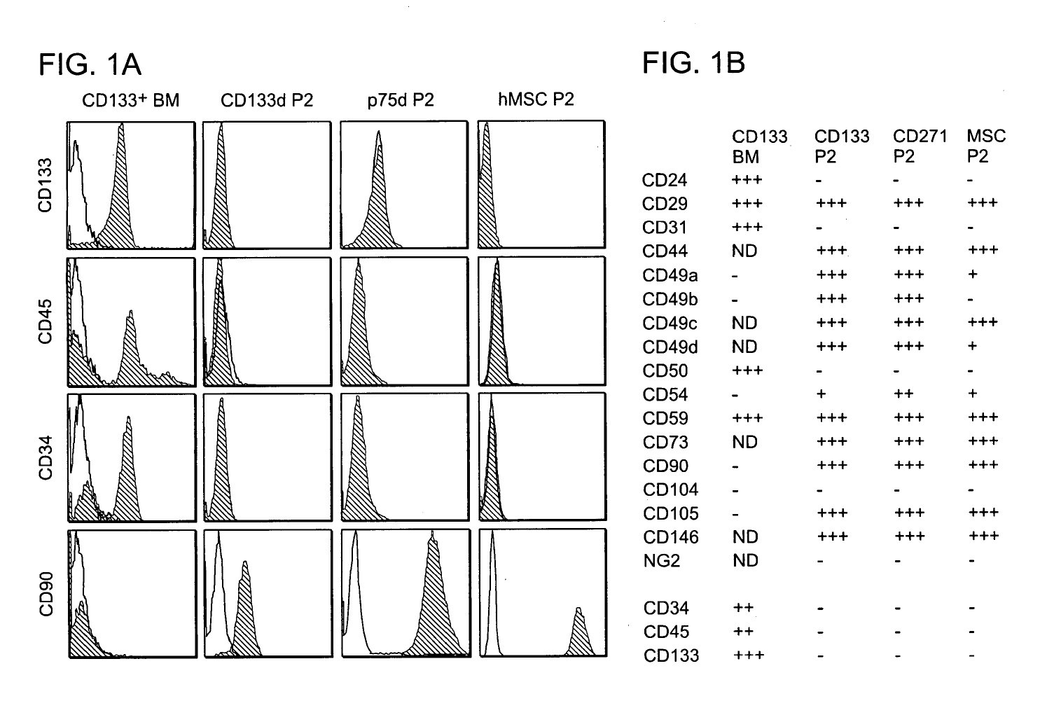

Analysis of cell surface epitopes demonstrated that the freshly isolated CD133⁺ cells were over 90% CD133⁺, 58% CD45⁺, 72% CD34⁺, 44% ABC G2⁺, 57% CD24⁺, and were negative for CD49a, CD49b, CD90, and CD105 (FIG. 1). The surface epitopes of the CD133⁺ cells and the p75LNGFR⁺ cells changed as they adhered and expanded to generate CD133dMSCs and p75dMSCs (FIG. 1). At passage 2 (P2), the CD133dMSCs were no longer positive for CD133, CD45, CD34, CD31, ABCG2 or CD24. Similarly, P2 p75dMSCs were no longer positive for the p75LNGFR epitope used to initially isolate the cells. P2 cultures of hMSCs, CD133dMSCs, and p75dMSCs were all negative for CD133, CD45, and CD34 (FIG. 1).

Expanding in serum-containing medium, all of the cells became positive for CD90 (Thy 1) and CD105 (Endoglin), epitopes that are characteristically expressed on hMSCs (FIG. 1). The CD133dMSCs and p75dMSCs also expressed CD29, CD44, and CD59, as did P2 hMSCs. The expanded CD133dMSCs and p75dMSCs became positive for the integrin epitopes CD49a and CD49b that are initially expressed on P0 hMSCs, but are lost as the hMSCs are expanded. Among the epitopes that were examined, none were found that could clearly distinguish between the cultured CD133dMSCs and the p75dMSCs. P2 hMSCs, CD133dMSCs, and p75dMSCs all expressed high levels of CD146 (MCAM), the recently described marker for the human non-hematopoietic bone marrow stem cell (FIG. 1).

### Example 2

### CD133dMSCs and p75dMSCs Readily Differentiated into Osteoblasts, Adipocytes, and Chondrocytes

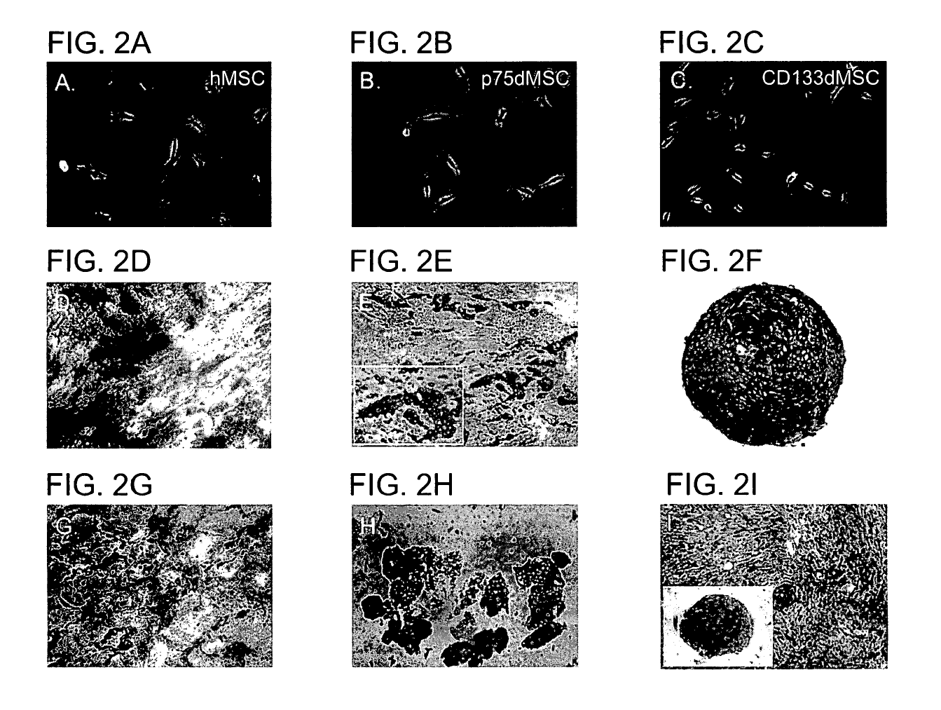

The hMSCs, CD133dMSCs, and p75dMSCs had similar morphologies during culture and through several passages (FIG. 2A, B, C). To assay the differentiation potential of the CD133dMSC and p75dMSC cultures, frozen vials of P1 and P2 cells were thawed, plated at 1,000 cells/cm², expanded for 5 days, and then transferred to medium to induce osteogenic, adipogenic, or chondrogenic differentiation. The CD133dMSCs and p75dMSCs readily differentiated into osteoblasts, adipocytes, and chondrocytes under the same culture conditions used to differentiate typical hMSCs (FIG. 2D-I).

### Example 3

### Growth Rates of hMSCs, CD133dMSCs, and p75dMSCs Under Normoxic and Hypoxic Conditions

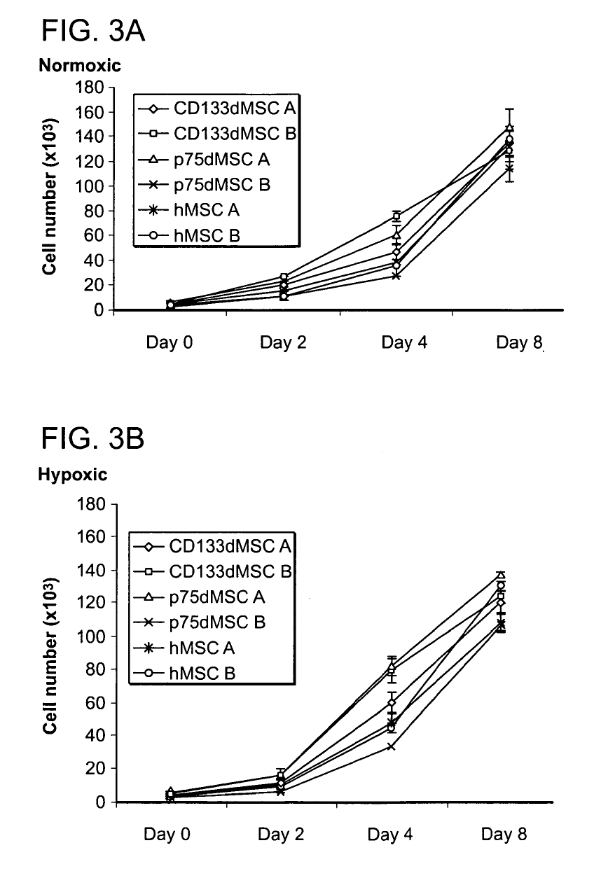

To determine the proliferative capacity of hMSCs, CD133dMSCs, and p75dMSCs under normoxic and hypoxic conditions (1% oxygen) cells were plated from two donors for each cell type in CCM (100 cells/cm²) and measured cell numbers on days 0, 2, 4 and 8. The hMSCs, CD133dMSCs, and p75dMSCs all grew equally well under normoxic and hypoxic conditions (FIGS. 3A and B).

### Example 4

### P2 CD133dMSCs and P2 p75dMSCs have Unique Gene Expression Profiles

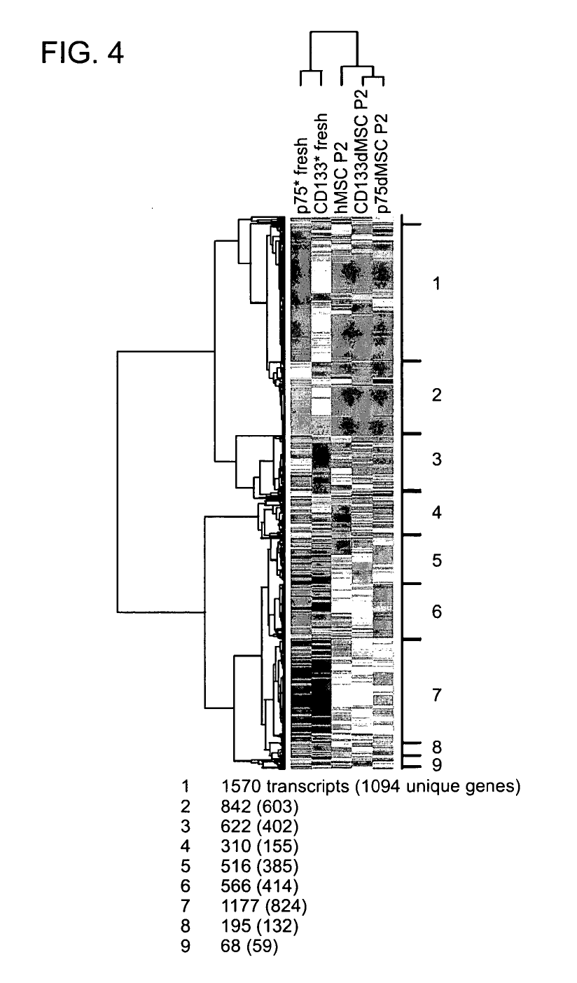

Hierarchical clustering of microarray data sets demonstrated that the transcriptional profiles of freshly isolated CD133-positive and CD271 (p75LNGFR)-positive cells from human bone marrow were more similar to each other than to P2 hMSCs, P2 CD133dMSCs or P2 p75dMSCs (see cluster diagram and heat map patterns 1 and 2; FIG. 4). Although sharing many expressed genes in common with P2 hMSCs (pattern 7, FIG. 4), several sets of differentially expressed genes demonstrated that the P2 CD133dMSCs and P2 p75dMSCs possessed unique gene expression profiles compared with typically-isolated hMSCs (patterns 4, 5, 6, 8, and 9; FIG. 4). Overall, the expressed genes from the CD133dMSCs and the p75dMSCs clustered together, apart from the hMSC profile, indicating that they were more similar to each other than to typical hMSCs (see cluster diagram; FIG. 4). Of interest, 3 major gene expression patterns identified upregulated sets of transcripts that were differentially expressed by hMSCs (pattern 4), CD133dMSCs (pattern 9), and p75dMSCs (pattern 6; FIG. 4, Table 1, below).

TABLE 1
Microarray GO terms
10 most significant GO terms for P2 hMSCs (pattern 4)

| | |
|---|---|
| cytoskeletal protein binding | p ≦ 0.00001 |
| cytoskeleton | p ≦ 0.0001 |
| actin binding | p ≦ 0.0001 |
| cell cycle checkpoint | p ≦ 0.001 |
| mitotic cell cycle checkpoint | p ≦ 0.001 |
| small GTPase regulator activity | p ≦ 0.001 |
| regulation of small GTPase mediated- | p ≦ 0.001 |
| signal transduction | |
| intercellular junction | p ≦ 0.001 |
| striated muscle contraction | p ≦ 0.001 |
| G1/S transition checkpoint | p ≦ 0.01 |

10 most significant GO terms for P2 CD133dMSCs (pattern 9)

| | |
|---|---|
| calcium ion transmembrane transporter- | p ≦ 0.001 |
| activity | |
| digestion | p ≦ 0.001 |
| hydrolase activity | p ≦ 0.001 |
| extracellular region | p ≦ 0.001 |
| extracellular region part | p ≦ 0.01 |
| phosphoric diester hydrolase activity | p ≦ 0.01 |
| collagen metabolic process | p ≦ 0.01 |
| extracellular matrix | p ≦ 0.01 |
| proteinaceous extracellular matrix | p ≦ 0.01 |
| growth factor activity | p ≦ 0.01 |

10 most significant GO terms for P2 p75dMSCs (pattern 6)

| | |
|---|---|
| anatomical structure development | p ≦ 0.0000001 |
| developmental process | p ≦ 0.0000001 |
| extracellular matrix | p ≦ 0.0000001 |
| extracellular matrix part | p ≦ 0.0000001 |
| proteinaceous extracellular matrix | p ≦ 0.0000001 |
| extracellular region | p ≦ 0.00001 |
| extracellular region par | p ≦ 0.00001 |
| anatomical structure morphogenesis | p ≦ 0.00001 |
| amine biosynthetic process | p ≦ 0.00001 |
| nitrogen compound biosynthetic process | p ≦ 0.00001 |

In addition, a cluster of expressed collagen genes was upregulated in the p75dMSCs that included Col3A1, Col4A1, Col5A1, Col7A1, Col11A1, and Col12A1; ‘Collagen’, p≦0.001. The complete G0 terms and associated transcripts for each heat map pattern from FIG. 4 are described below.

### Example 5

### Growth Factor and Cytokine Secretion Responses

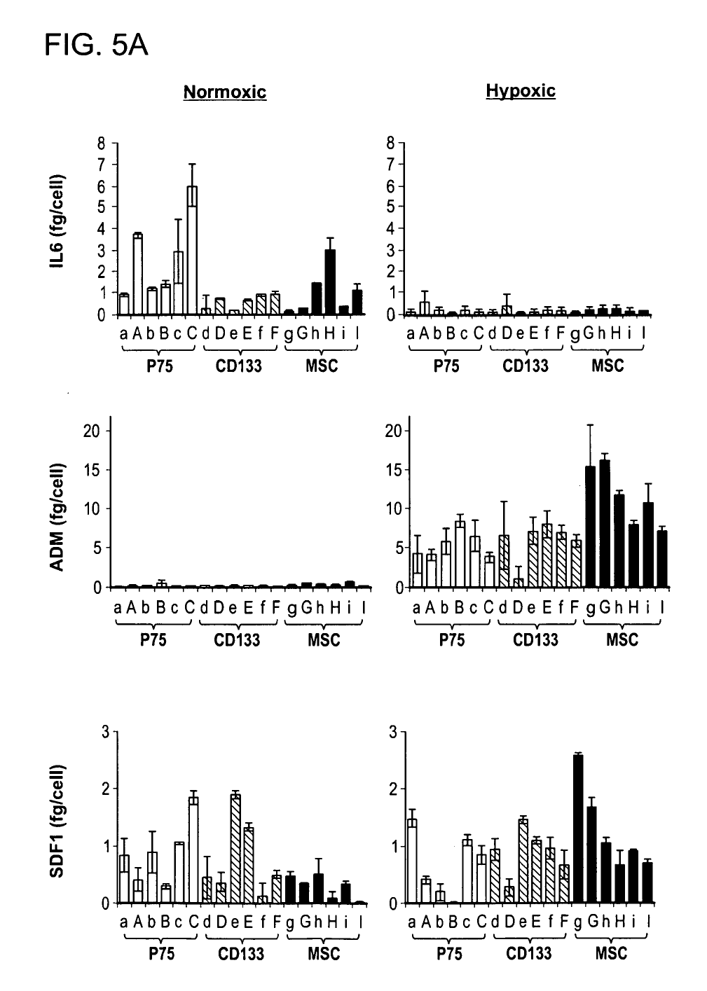

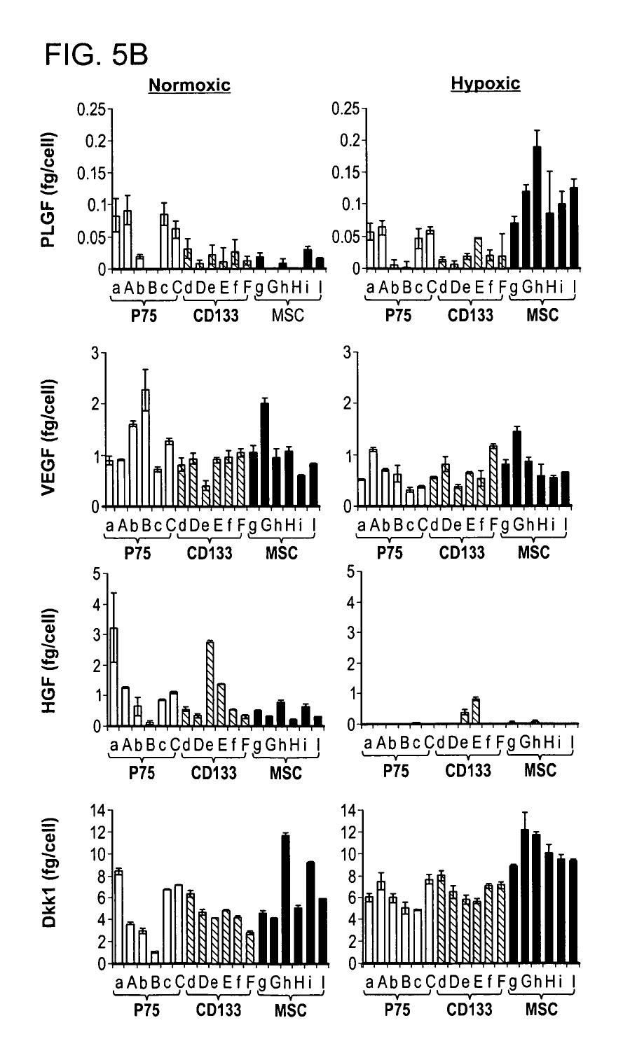

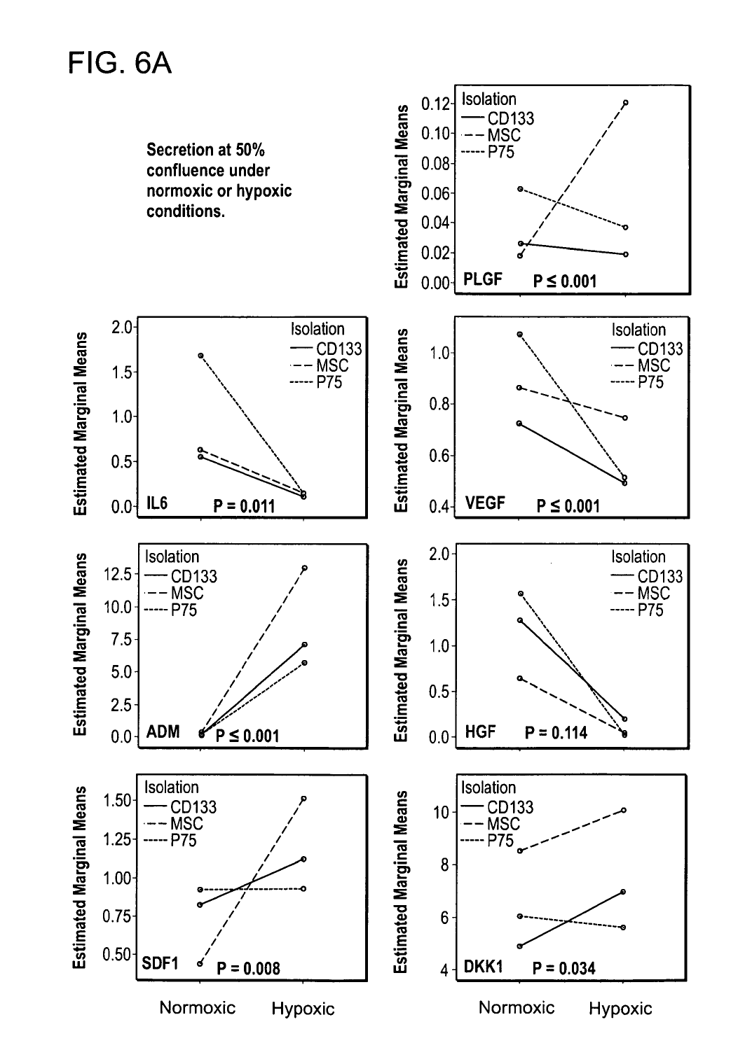

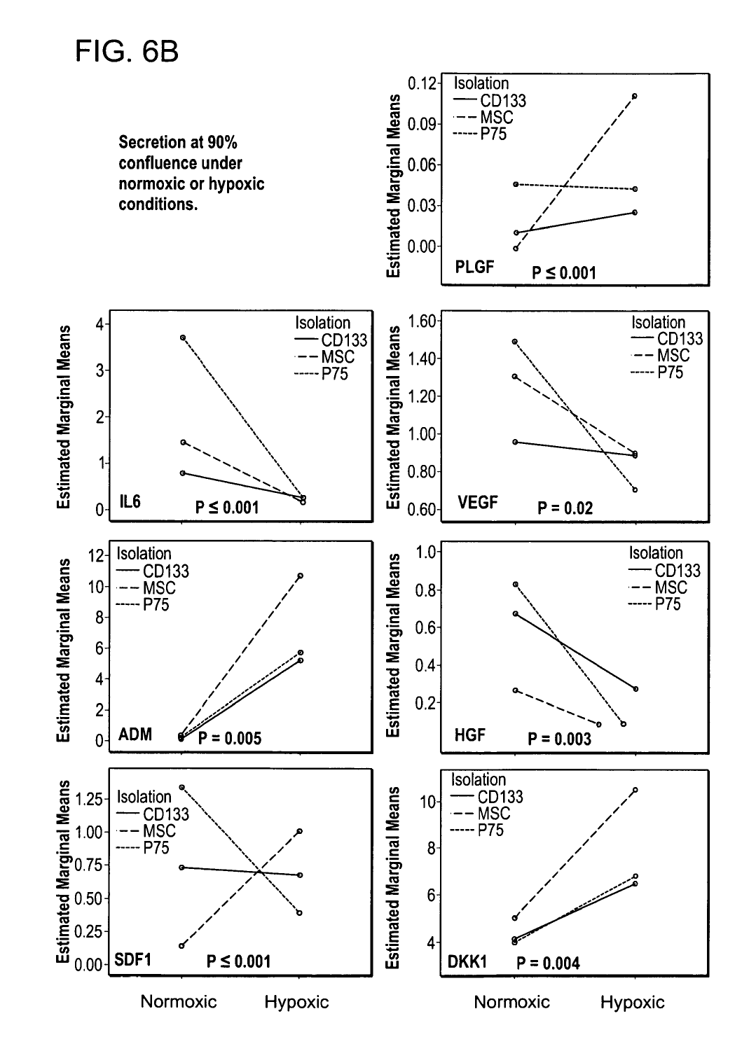

To examine the secretion responses of hMSCs, CD133dMSCs and p75dMSCs for selected growth factors and cytokines, ELISAs were run on mediums conditioned by each cell type for 48 hours at 50% and 90% cell confluence and under normoxic or hypoxic conditions (FIG. 5). Significant differences in protein/peptide secretion between the three progenitor cell populations were determined by repeated measures analysis of variance (ANOVA). Analysis of estimated marginal means for protein/peptide secretion on a per cell basis demonstrated that the three progenitor cell populations responded in a significantly different manner to hypoxia exposure at both 50% and 90% cell confluence (50%, IL6, p=0.011; ADM, p≦0.001; SDF1, p=0.008; PLGF, p≦0.001; VEGF, p≦0.001; Dkk1, p=0.034; and 90%, IL6, p≦0.001; ADM, p=0.005; SDF1, p≦0.001; PLGF, p≦0.001; VEGF, p=0.020; HGF, p=0.003; Dkk1, p=0.004 (FIGS. 6A and B). The response for HGF secretion did not differ between the 3 progenitor cell populations at 50% confluence (HGF, p=0.114; FIG. 6A). Under the conditions that were used to generate MSC CdM, the secretion of Epidermal Growth Factor (EGF), Basic Fibroblast Growth Factor (bFGF), and Platelet-Derived Growth Factor-AB (PDGF-AB) was beyond the limits of detection (<2 pg/ml), and the secreted levels of Beta Nerve Growth Factor (β-NGF) and Leukemia Inhibitor Factor (LIF) were low (<30 pg/ml). Bonferroni pairwise comparisons were used to determine differences between the cell populations in their levels of particular secreted factors at a given cell confluence and under normoxic or hypoxic conditions.

### Interleukin 6 (IL6)

Secreted IL6 regulates HSC numbers and is also expressed and released from tissues during inflammatory responses. Under normoxic conditions, the p75dMSCs secreted significantly greater amounts of IL6 than did either the CD133dMSCs or the hMSCs (50%, vs. CD133dMSC, p=0.022; 50%, vs. MSC, p=0.034; FIG. 6A) (90%, vs. CD133dMSC, p≦0.001; 90%, vs. MSC, p=0.006; FIG. 6B). IL6 secretion levels for the hMSCs and CD133dMSCs under normoxic conditions were not significantly different. At 50% confluence under hypoxic conditions, the levels of secreted IL6 were not significantly different for hMSCs or CD133dMSCs compared with their secreted levels under normoxia. In contrast, at 50% confluence under hypoxic conditions, the p75dMSCs significantly decreased their secretion of IL-6 (10.8 fold decrease, p≦0.001; FIG. 6A). At 90% confluence under hypoxic conditions, secretion of IL6 by CD133dMSCs was not different from IL6 secretion under normoxia, while IL6 secretion from hMSCs and p75dMSCs significantly decreased (hMSC, 8.7 fold decrease, p=0.01; p75dMSC, 14.9 fold decrease, p≦0.001; FIG. 6B).

### Adrenomedullin (ADM)

ADM is a secreted vasodilating peptide that acts to reduce cellular oxidative stress and apoptosis. ADM secretion was significantly increased in all of the progenitor cell populations under hypoxic conditions compared with their secretion levels under normoxic conditions, regardless of cell density (50%, CD133dMSC, 42.7 fold increase, p≦0.001; hMSC, 30.9 fold increase, p≦0.001; p75dMSC, 20.3 fold increase, p≦0.001; FIG. 6A) (90%, CD133dMSC, 33.2 fold increase, p≦0.001; hMSC, 29.9 fold increase, p≦0.001; p75dMSC, 22.0 fold increase, p≦0.001; FIG. 6B). Under hypoxic conditions, ADM secretion by CD133dMSCs and p75MSCs was not significantly different, while the hMSCs secreted significantly higher levels of ADM than did the other cell types at either cell density (50%, vs. CD133dMSC, p≦0.001; vs. p75dMSC, p≦0.001; FIG. 6A) (90%, vs. CD133dMSC, p=0.009; vs. p75dMSC, p=0.017; FIG. 6B).

### Vascular Endothelial Growth Factor (VEGF)

VEGF has numerous biological effects that include angiogenesis, cellular protection, and mobilization of bone marrow-derived cells. In the studies described herein, VEGF secretion generally decreased for all three progenitor cell populations under hypoxic conditions as compared with secretion under normoxic conditions (50%, CD133dMSC, 1.5 fold decrease, p=0.002; hMSC, NS; p75dMSC, 2.1 fold decrease, p≦0.001; FIG. 6A) (90%, CD133dMSC, NS; hMSC, 1.5 fold decrease, p=0.021; p75dMSC, 2.1 fold decrease, p≦0.001; FIG. 6B). Under hypoxic conditions at 50% confluence, the hMSCs secreted significantly more VEGF than did either other cell type (vs. CD133dMSC, p=0.006; vs p75dMSC, p=0.011; FIG. 6A). At 90% confluence, however, none of the populations differed significantly from each other in terms of VEGF secretion.

### PLacental Growth Factor (PLGF)

PLGF is a VEGF family member that binds VEGFR1 and functions in pathological angiogenesis.³⁹ Under normoxia at 50% confluence, the p75dMSCs secreted significantly greater levels of PLGF than did either the CD133dMSCs or the hMSCs (50%, vs. CD133dMSC, p=0.009; vs. hMSC, p=0.001; FIG. 6A). However, the hMSCs demonstrated a dramatic increase in PLGF secretion in response to hypoxia compared with the responses of the other cell types (50%, hMSC, 6.72 fold increase, p≦0.001; CD133dMSC, NS; p75dMSC, NS; FIG. 6A) (90%, hMSC, 12.9 fold increase, p≦0.001; CD133dMSC, NS; p75dMSC, 1.4 fold decrease, p=0.043; FIG. 6B). Under hypoxic conditions, hMSCs secreted greater levels of PLGF than did either other cell type (50%, vs. CD133dMSC, p≦0.001; vs. p75dMSC, 0.001; FIG. 6A) (90%, vs. CD133dMSC, p≦0.001; vs. p75dMSC, p≦0.001; FIG. 6B). The secreted levels of PLGF did not differ between CD133dMSCs and p75dMSCs under hypoxic conditions at either cell density tested.

### Dickkopf-1 (DKK1)

DKK1 is a negative regulator of Wnt signaling and functions in a paracrine manner to regulate MSC entry into the cell cycle. In general, DKK1 secretion for all three cell populations increased in response to hypoxia exposure regardless of cell density (50%, CD133dMSC, 1.4 fold increase, p=0.005; hMSC, 1.2 fold increase, p=0.028; p75dMSC, NS; FIG. 6A) (90%, CD133dMSC, 1.6 fold increase, p≦0.001; hMSC, 2.1 fold increase, p≦0.001; p75dMSC, 1.7 fold increase, p≦0.001; FIG. 6B). Under hypoxic conditions, the hMSCs secreted significantly greater levels of DKK1 than did either other cell type (50%, vs. CD133dMSC, p≦0.001; vs. p75dMSC, p≦0.001; FIG. 6A) (90%, vs. CD133dMSC, p≦0.001; vs. p75dMSC, p≦0.001; FIG. 6B). Under hypoxia at 50% confluence, the CD133dMSCs secreted more DKK1 that did the p75dMSCs (p=0.03), however the CD133dMSC and p75dMSC subpopulations did not differ in DKK1 secretion levels for the other conditions tested.

### Stromal Derived Factor 1 (SDF1)

SDF1 controls in part HSC retention within and migration out of the bone marrow microenvironment. The secretion responses for SDF1 clearly differed between the hMSCs and the epitope-isolated subpopulations. Under hypoxia at 50% confluence, the hMSCs significantly increased their SDF1 secretion (3.4 fold increase, p≦0.001) while the other two cell populations did not significantly alter their levels of SDF1 secretion (FIG. 6A). Under hypoxia at 90% confluence, the hMSCs also increased their SDF1 secretion (6.95 fold increase, p=0.005), while the CD133dMSCs did not significantly alter their SDF1 secretion levels and the p75dMSCs actually decreased their SDF1 secretion (3.4 fold decrease, p=0.002; FIG. 6B).

### Hepatocyte Growth Factor (HGF)

HGF has diverse roles in angiogenesis, cell survival, and cancer cell metastasis. HGF secretion generally decreased in response to hypoxia for all three cell populations regardless of cell density (50%, CD133dMSC, 6.3 fold decrease, p=0.002; hMSC, NS; p75dMSC, 71.6 fold decrease, p≦0.001; FIG. 6A) (90%, CD133dMSC, 2.5 fold decrease, p≦0.001; hMSC, 26.6 fold decrease, p=0.024; p75dMSC, 165 fold decrease, p≦0.001; FIG. 6B). At 90% confluence under normoxic conditions, the p75dMSCs secreted significantly more HGF that did the hMSCs (p=0.037), but did not differ significantly in HGF secretion compared with that of the CD133dMSCs at either cell density (FIGS. 6A and B).

### Example 6

### Effects of CD133dMSCs and CD133dMSC CdM Following Cerebral Ischemia

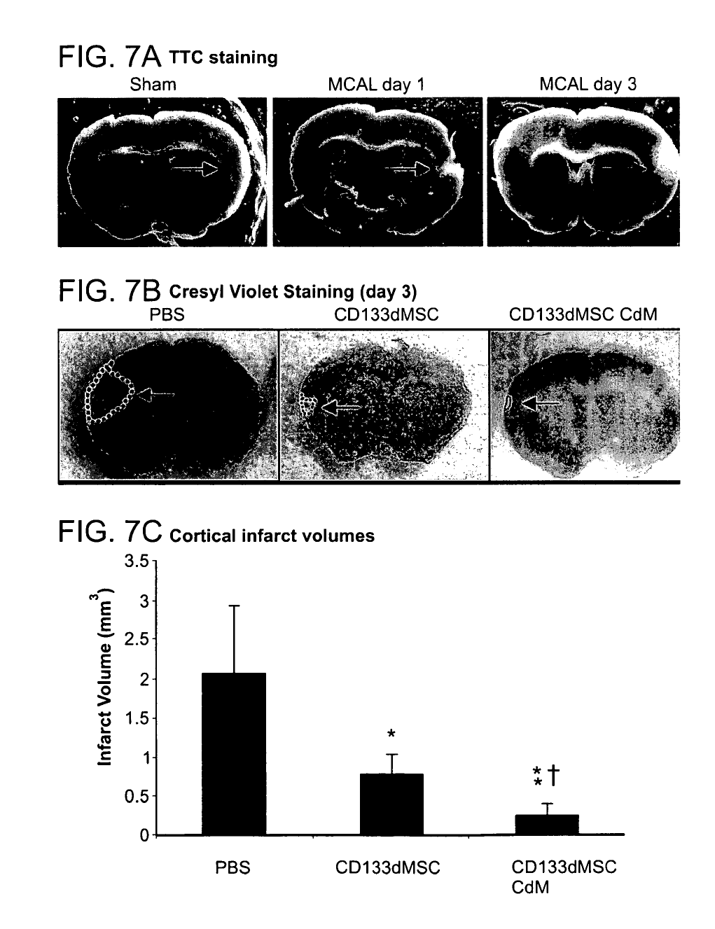

To determine whether the factors secreted by an epitope-isolated subpopulation would provide benefits in the context of tissue injury, CD133dMSCs or concentrated CD133dMSC CdM were administered to immunodeficient mice one day after permanent ligation of the middle cerebral artery. Intracardiac (arterial, left ventricle) administration of either cells or CdM protected against the effects of cerebral ischemia and significantly reduced cortical infarct volumes (PBS, 2.1±0.86 mm³, n=5; CD133dMSCs, 0.77±0.26 mm³, n=6, p=0.026; CD133dMSC CdM, 0.25±0.15 mm³, n=6, p=0.009; FIG. 7). A single administration of concentrated CD133dMSC CdM provided superior protection compared with cell injection (p=0.003, FIG. 7).

The results described herein demonstrate that human bone marrow contains different subpopulations of multipotent non-hematopoietic progenitor cells that can be enriched on the basis of CD133 or CD271 expression. The transcriptional profiling and protein secretion data show that both subpopulations are significantly different from the total adherent transit-amplifying progenitor cells that are commonly denoted “MSCs”. Magnetic sorting against CD133 directly isolates multipotent MSC-like cells from human bone marrow, indicating that CD133 is likely to be expressed by non-hematopoietic bone marrow stems in vivo (Perry et al., Cytotherapy. 2005; 7: 89). CD133 is expressed by adult stein/progenitor cells from many tissues including HSCs and hemangioblasts, endothelial progenitor cells, liver stem cells, pancreatic stem cells, neural stem cells, and stem-like cancer initiating cells. Mutations in CD133 (PROM1) lead to photoreceptor disk malformations and macular degeneration in patients, (Yang et al. J Clin Invest. 2008; 118:2908-2916), although the precise function of CD133 for stem/progenitor cells is unknown.

CD271 (p75LNGFR) functions in pan-neurotrophin signaling during development and is expressed by germline stem cells (Nykjaer et al., Curr Opin Neurobiol. 2005; 15:49-57; Robinson et al., J Clin Endocrinol Metab. 2003; 88:3943-3951. In adults, the p75LNGFR is expressed by several types of stem/progenitor cells including keratinocyte stem cells and neural stem cells (Nakamura et al., Stem Cells. 2007; 25:628-638; Young et al., J Neurosci. 2007; 27:5146-5155). Knockout mice for this receptor have vascular defects (Kraemer et al., Circ Res. 2002; 91:494-500). Immunohistochemical assays using antibodies against p75LNGFR were initially reported to stain recticular cells in sections of human bone (Cattoretti et al., Blood. 1993; 81:1726-1738). Subsequent studies used magnetic sorting against the p75LNGFR to isolate adherent MSC-like cells that expanded in culture and differentiated into osteoblasts and adipocytes (Quirici et al., Exp Hematol. 2002; 30:783-791). As reported herein, expanded p75dMSCs differentiates into osteoblasts, adipocytes, and chondrocytes under the same culture conditions used to differentiate hMSCs and CD133dMSCs.

Most of the cell surface markers commonly used to describe the “MSC” phenotype were shared between P2 hMSCs, CD133dMSCs and p75dMSCs. However, based on CD49a and CD49b expression, early passage CD133dMSC and p75dMSC cells likely contain a higher percentage (e.g., 10%, 25%, 50%, 75% higher) of stem-like progenitor cells than do typical hMSCs of the same passage. Early passage hMSCs (P1) are reported to express both CD49a and CD49b (Delorme et al. Blood. 2008; 111:2631-2635), but these epitopes appear to be expressed at reduced levels at later passages. As shown herein, higher percentages of P2 CD133dMSCs and p75dMSCs expressed CD49a than did P2 hMSCs. Furthermore, the majority of P2 CD133dMSCs and P2 p75dMSCs expressed high levels of CD49b, an epitope that was not expressed by P2 hMSCs. Several other groups have described additional epitopes that prospectively isolate hMSCs from bone marrow with varying degrees of efficiency including CD49b, CD90, CD105 (low efficiency) (Delorme et al. Blood. 2008; 111:2631-2635), STRO-1 (high efficiency) (Gronthos et al., Blood. 1994; 84:4164-4173), CD73, CD130, CD146, CD200, integrin alphaV/beta5 (high efficiency) (Delorme et al. Blood. 2008; 111:2631-2635), and also CD140b, CD340, and CD349 (Bühring et al., Ann N Y Acad Sci. 2007; 1106:262-271). Sacchetti et al. reported recently that CD146 (MCAM) expression identified cell populations from human bone marrow that contained a non-hematopoietic stem cell capable of self-renewal and of providing an ectopic hematopoietic microenvironment (HME) in mice (Sacchetti et al., Cell. 2007; 131: 324-336). In addition, CD146-positive cells re-isolated from the primary ectopic HME could be expanded and used to transfer a second ectopic HME to a different animal (an indication of stem cell activity). They found that bone marrow osteoblasts and dermal fibroblasts did not express the CD146 epitope. All of the hMSCs, CD133dMSCs, and p75dMSCs used in the studies reported herein expressed high levels of CD146. Based on the identified multi-potentiality and expression of CD146, it is likely that each of these cell populations contains some self-renewing non-hematopoietic stem-like cells. As secreted growth factors and cytokines, such as IL6 play critical roles in regulating hematopoiesis, the ELISA results described above indicate that functional differences between typically-isolated hMSCs and the CD133dMSC and p75dMSC subpopulations exist.

Plastic adherent hMSCs adopt distinct morphologies in low density culture conditions that are distinguished by their rate of expansion and also by their differentiation potential; small rapidly self-renewing MSCs (RS cells) and larger slowly-replicating MSCs (SR cells) (Sekiya et al., Stem Cells. 2002; 20: 530-541; Colter et al., Proc. Natl. Acad. Sci. USA, 2001; 98:7841-7845; Lee et al., Blood. 2006; 107:2153-2161). In culture, the RS cells give rise to intermediate-sized hMSC phenotypes and to the SR cells) (Sekiya et al., Stem Cells. 2002; 20: 530-541; Digirolamo et al., Br J Haematol. 1999; 107:275-281) suggesting that the emergence of these two cell types may reflect growth and commitment to various precursor cells in serum-containing expansion mediums. The RS and SR cells appear to possess different properties that could potentially be exploited in cell-based therapies (Lee et al., Blood. 2006; 107:2153-2161). Similar to typical hMSC cultures, similar RS and SR cell morphologies and population dynamics were observed in cultures of CD133dMSCs and p75dMSCs. Importantly, however, despite possible clonal variation within the cultures, hMSCs, CD133dMSCs and p75dMSCs maintained significant differences at the level of transcription at P2 and at the level of protein/peptide secretion at P5. Crigler et al. reported heterogeneity in the secreted levels of BDNF and NGF in clonal single cell-derived subpopulations of human MSCs (Crigler et al., Exp Neurol. 2006; 198:54-64). Cells isolated in this manner are likely, for example, to be used to identify useful cell surface epitopes for the prospective isolation of hMSCs with a particular secretory phenotype.

To determine whether the secreted factors from an epitope-isolated human non-hematopoietic progenitor subpopulation would provide benefits in the context of ischemic tissue injury, CD133dMSCs and CD133dMSC conditioned media was administered to immunodeficient mice with cerebral ischemia. Both the cells and the CdM provided significant protection against the injury as demonstrated by dramatically reduced cortical infarct volumes.

### Example 7

### Isolation of Bone Marrow Cells Expressing p75LNGFR

The heart is an important target for tissue repair because of the prevalence of heart disease, the limited capacity for the heart to repair itself, and the challenge associated with obtaining biopsy material to prepare adult stem/progenitors for cell therapy. Recently, it was shown that MSC treatment improved cardiac function after myocardial infarction (MI) in part through paracrine action or independently of long-term engraftment (Zimmet et al., Basic Res Cardiol 2005; 100:471-481; Noiseux et al., Mol Ther 2006; 14:840-850; Gnecchi et al., FASEBJ 2006; 20:661-669; Iso et al., Biochem Biophys Res Commun 2007; 354:700-708). Conditioned medium from MSCs has previously been shown to protect cardiomyocytes from cell death (Gnecchi et al., FASEBJ 2006; 20:661-669; Iso et al., Biochem Biophys Res Commun 2007; 354:700-708), however, the effects of MSC-secreted factors on adult cardiac stem/progenitor cells (CSCs/CPCs) was unknown. The results described below were obtained to determine whether factors secreted from adult bone marrow MSCs would affect the growth and survival of adult CSCs/Cardiac progenitor cells.

In the bone marrow compartment one of the cell types produced by MSCs, the stromal cell, is known to support the growth and differentiation of hematopoietic stem cells (HSCs) by providing critical niche components. The niche components include both cellular substrate, e.g. extracellular matrix, as well as multiple secreted factors such as cytokines and growth factors that influence HSC growth, survival, and function. In the bone marrow, MSCs localize along the endosteal surface of the bone (an HSC niche) and also in a vascular-associated niche. HSCs co-exist in locations within the bone marrow where the supportive MSCs and the MSC-derived stromal cells are found. In terms of influencing endogenous tissue stem/progenitor cells during repair processes, factors produced by supportive cells such as mesenchymal cells, astrocytes, or endothelial cells may be especially pertinent to examine because these cell types form the supportive elements of many adult stem cell niches.

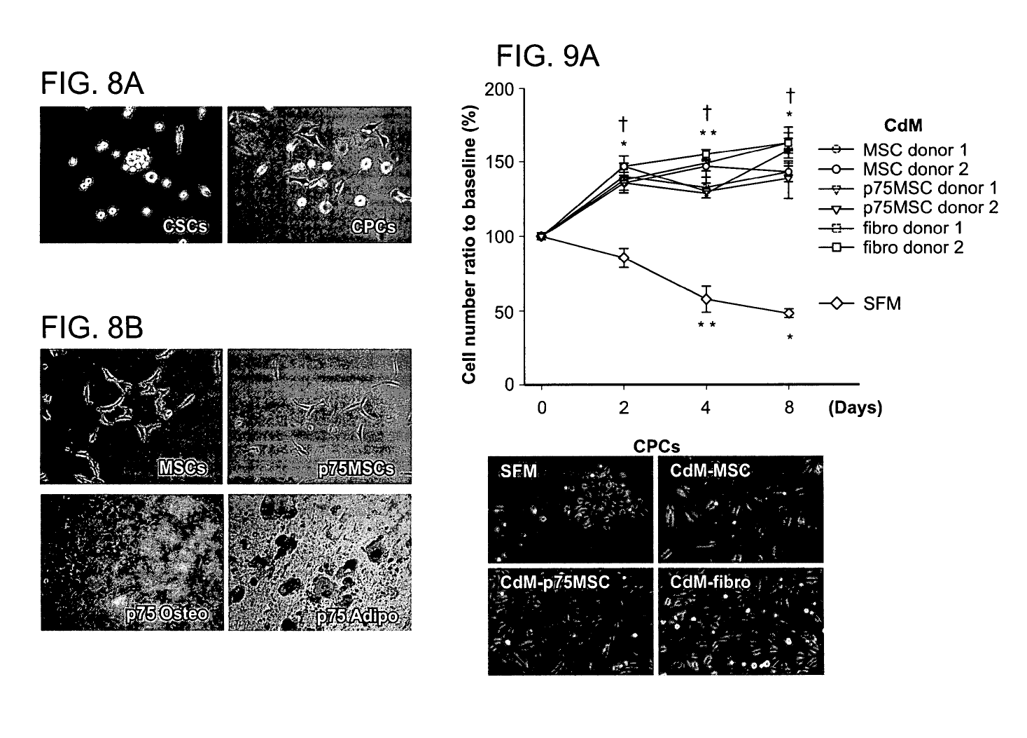

MSCs are typically isolated from bone marrow by discontinuous density gradient centrifugation. The mononuclear cell layer is cultured and the MSCs are isolated by their adherence to the culture plastic after 24-48 hrs. MSCs are then propagated for 7-10 days. The p75MSC subpopulation was isolated from bone marrow mononuclear cells with the use of magnetic selection for p75LNGFR. The isolated bone marrow cells that expressed p75LNGFR adhered to plastic culture dishes and propagated in a manner similar to non-selected MSCs. By FACS analysis, similar to non-magnetically selected MSCs, the p75MSCs expressed CD44, CD90 and CD105 and were negative for CD31, CD34, and CD45. The p75MSCs readily generated single cell-derived colonies, had a fibroblastic spindle-like shape typical of MSCs, and differentiated into osteogenic cells that stained with Alizarin red S and adipogenic cells that stained with Oil red O when exposed to the appropriate differentiation media (FIGS. 8A and 8B).

### Example 8

### Condioned Media Induced Cardiac Progenitor Cell Proliferation

Serum-free condioned media was collected from MSCs and p75MSCs to determine whether factors secreted by hMSCs would affect the growth of cardiac progenitor cell. Condioned media derived from fibroblasts was used as a positive control because fibroblasts are well known to support the growth of embryonic stem cells and various adult stem cell subtypes including cardiac stem cells when used as feeder layers (Quirici et al., Exp Hematol 2002; 30:783-791; Cattoretti et al., Blood 1993; 81:1726-1738; Gregory et al., Exp Cell Res 2005; 306:330-335; Beltrami et al., Cell 2003; 114:763-776; Dawn et al., Proc Natl Acad Sci USA 2005; 102:3766-3771; Richards et al., Nat Biotechnol 2002; 20:933-936).

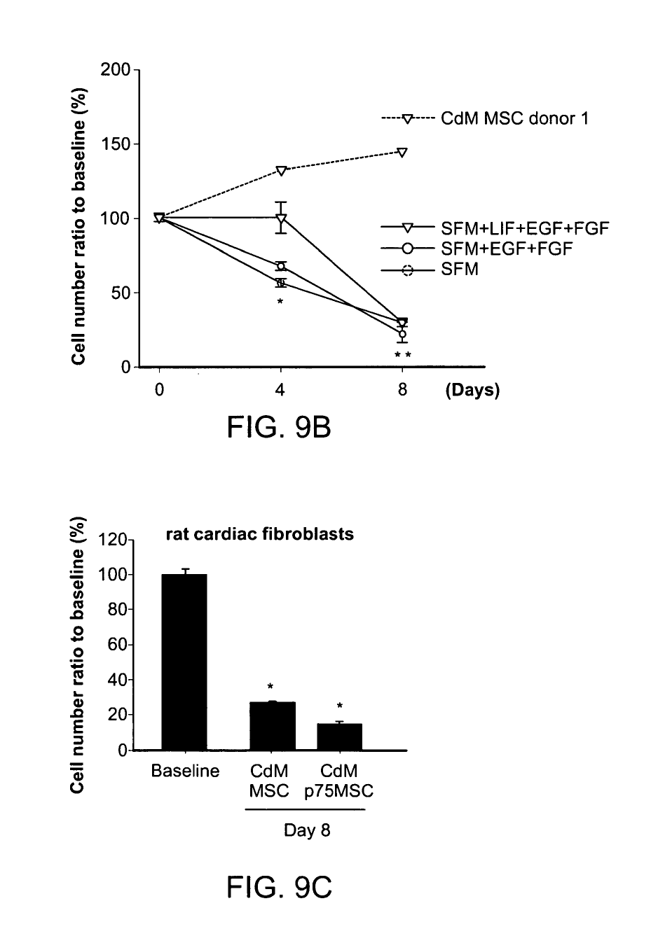

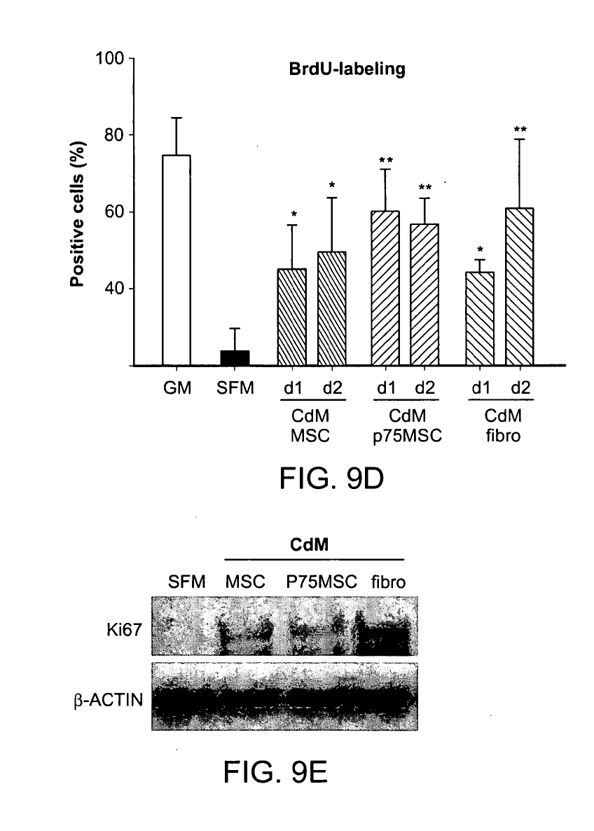

In the presence of condioned media from MSCs, p75MSCs or fibroblasts, the cardiac progenitor cells appeared to be more “differentiated” in morphology and were larger in size than those grown in the growth medium (FIG. 9A). Time course proliferation assays demonstrated that conditioned media from each of the cell types significantly induced the proliferation of cardiac progenitor cells while the number of cardiac progenitor cells incubated in serum-free medium (α-MEM) gradually decreased (FIG. 9A). Significant differences in cell numbers were maintained and expanded between the serum-free medium-treated and the conditioned media-treated cardiac progenitor cells throughout the experimental period. Thus, factors secreted by hMSCs induced proliferation of cardiac progenitor cells.

In contrast, neither serum-free α-MEM supplemented with 10 ng/ml of EGF and bFGF nor with 10 ng/ml of LIF, EGF and bFGF propagated cardiac progenitor cells (FIG. 9B). The number of cardiac progenitor cells incubated in these conditions significantly decreased at day 8 compared with the baseline as well as in fresh serum-free medium. Interestingly, conditioned media from MSCs and p75MSCs did not support the proliferation of adult rat cardiac fibroblasts (FIG. 9C)

To confirm DNA synthesis and active cell cycle status, incorporation of BrdU in cardiac progenitor cells was quantified after 24 hours of exposure to the conditioned media. The percentage of BrdU-positive cardiac progenitor cells incubated in the conditioned media from MSCs, p75MSCs or fibroblasts was significantly greater than that of serum-free medium-treated cells (FIG. 9D). Immunoblotting demonstrated that Ki67 was expressed in cardiac progenitor cells treated with conditioned media, but not in Cardiac progenitor cells treated with serum-free medium (FIG. 9E).

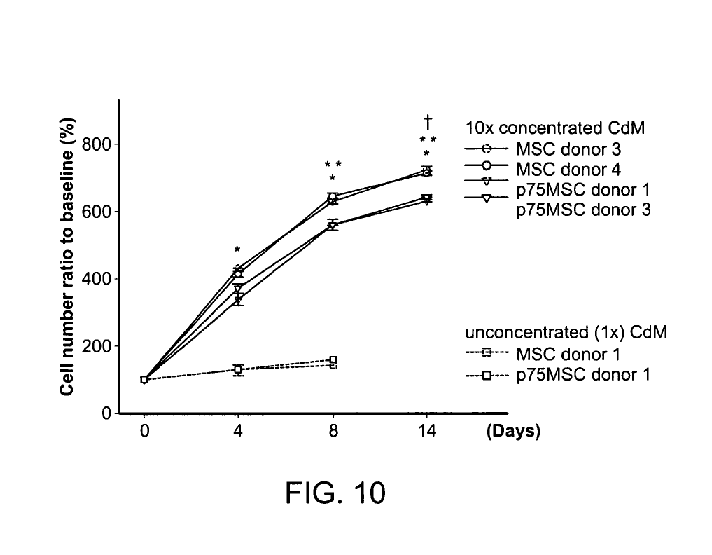

Furthermore, there was a concentration-dependent increase in cardiac progenitor cell number when the cardiac progenitor cells were incubated in 10×-concentrated conditioned media from MSCs and p75MSCs (FIG. 10). Cardiac progenitor cells treated with 10×-concentrated conditioned media continued to grow at least until 14 days after the initial 10×-conditioned media exposure. The cardiac progenitor cell number at day 14 in each 10×-conditioned media was significantly higher than that of the earlier time points and about a 6-7 fold increase in cardiac progenitor cell number from baseline (FIG. 10).

### Example 9

### Conditioned Media Activates STAT3 in Cardiac Progenitor Cells

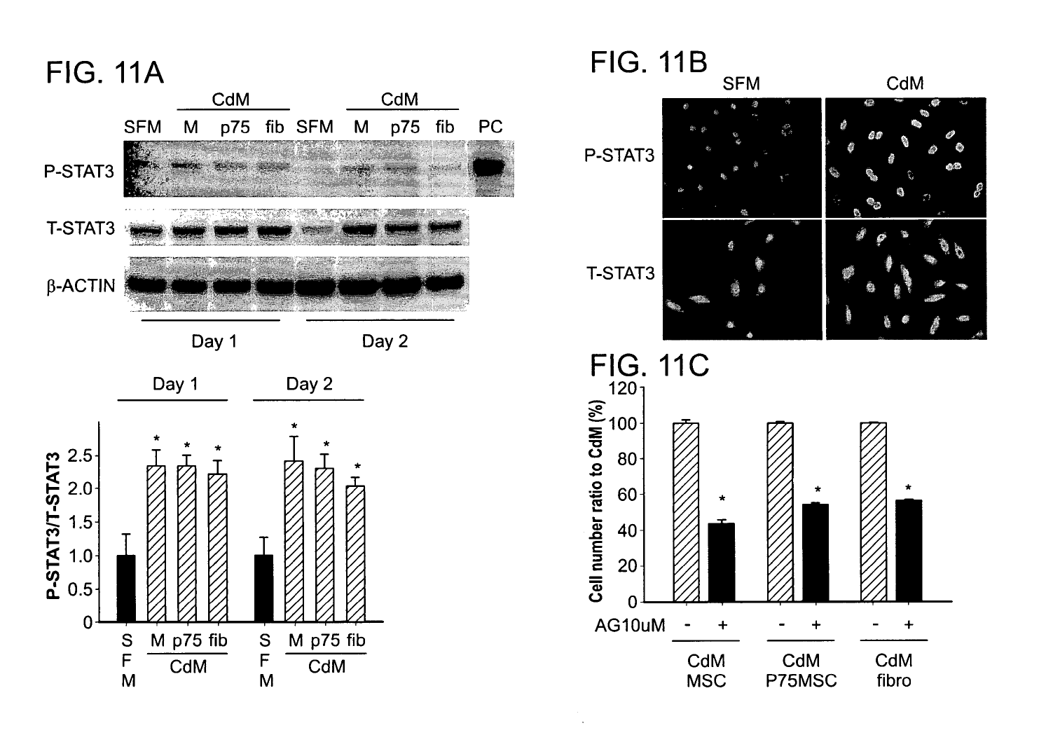

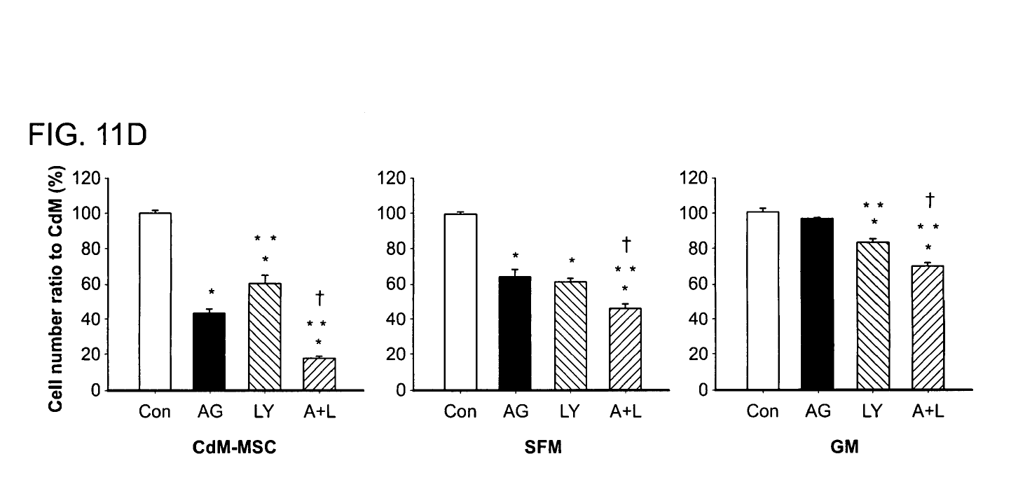

Phosphorylation of STAT3 (Tyr⁷⁰⁵) was detected 1 and 2 days after exposure of cardiac progenitor cells to conditioned media from MSCs, p75MSCs, and fibroblasts, and the phosphorylation levels in cardiac progenitor cells treated with conditioned media were significantly higher than in progenitor cells after exposure to serum-free medium (FIG. 11A). Immunocytochemistry demonstrated that the phosphorylated STAT3 was localized to the nuclei of the cardiac progenitor cells treated with conditioned media (FIG. 11B).

To determine whether phosphorylation of STAT3 mediated the effects of conditioned media on CSC activation, the Jak2/STAT3 pathway inhibitor AG490 was used. AG490 reduced the number of cardiac progenitor cells treated with conditioned media from the MSCs in a dose-dependent manner: control (CdM+DMSO), 100±1.5%; 1 μM, 96.3±0.9%; 5 μM, 89.5±0.9%; 10 μM, 43.4±2.4% (cell number ratio to control cell number (121,863 cells), mean±SEM, n=3 to 6). The inhibitory effect of AG490 10 μM was also observed in cardiac progenitor cells treated with the conditioned media from p75MSCs and fibroblasts (FIG. 11C). LY294002, the PI3K/Akt pathway inhibitor, also decreased the number of cardiac progenitor cells treated with the MSC Conditioned media, but to a lesser extent than AG490 (FIG. 11D). PD98059, the ERK inhibitor did not block the positive growth effects of the conditioned media (data not shown).

Although AG490 significantly reduced the number of cardiac progenitor cells incubated in serum-free medium, the reduction rate of the AG490 treatment was nearly equal to the LY294002 treatment (FIG. 11D). Of interest, LY294002, but not AG490 significantly diminished CPC numbers in growth medium, indicating that the factors promoting CPC growth in growth medium differ from the active factors in conditioned media (predominantly STAT3-activating). The combination of AG490 with LY294002 significantly decreased cardiac progenitor cell numbers in conditioned media, serum-free medium, and growth medium compared with control numbers. The combined inhibitors were most effective in reducing the numbers of cardiac progenitor cells in conditioned media (FIG. 11D).

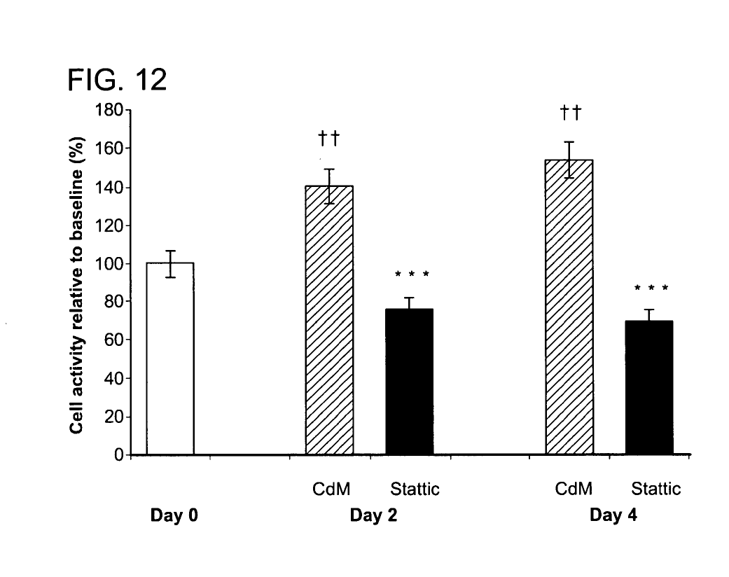

To confirm that secreted factors in MSC Conditioned media signaled through Jak2 and then through STAT3 in Cardiac progenitor cells, a new specific inhibitor of STAT3 was used (STAT3 Inhibitory Compound, aka Stattic) that blocks the phosphorylation of STAT3 at Tyr⁷⁰⁵ but does not block the phosphorylation of Jak1, Jak2, or STAT1. The inhibitor Stattic completely blocked the growth of cardiac progenitor cells in MSC conditioned media demonstrating that STAT3 is the critical proliferation-inducing transcription factor that is activated in cardiac progenitor cells by MSC conditioned media (FIG. 12). Thus, these data indicate that activation of STAT3 mediates in part the proliferation and survival of cardiac progenitor cells induced by conditioned media from hMSCs.

### Example 10

### Conditioned Media-Expanded Cardiac Progenitor Cells are Multipotent

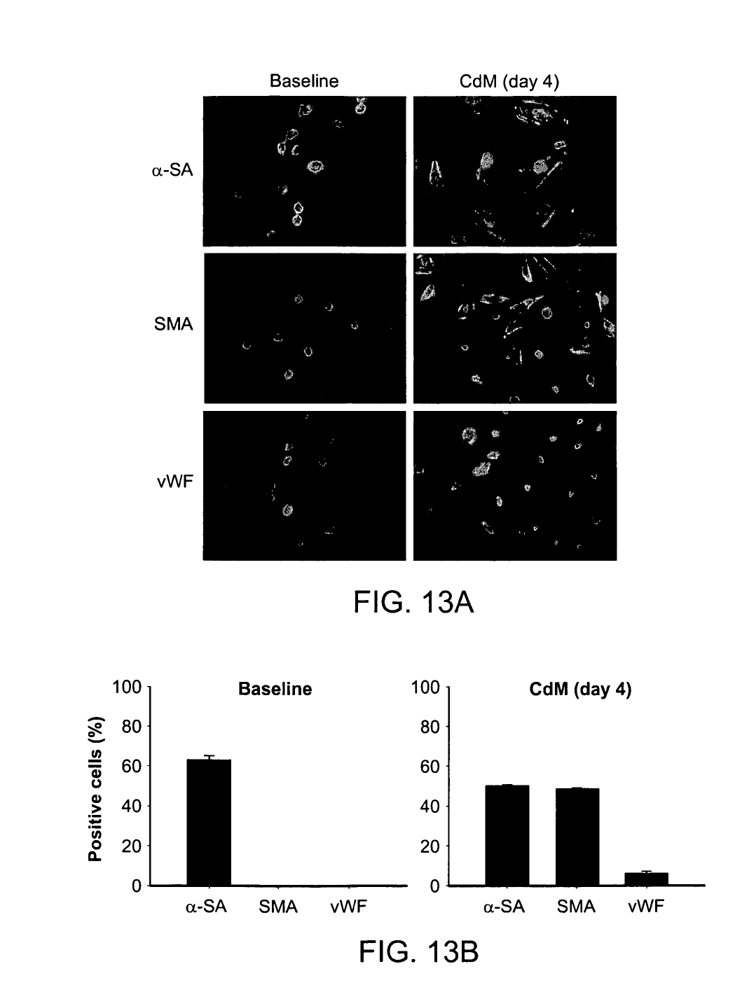

Because cardiac progenitor cells incubated in conditioned media were flatter and larger than those incubated in growth medium, immunocytochemistry was used to determine whether cardiac progenitor cells grown in conditioned media exhibited evidence of differentiation. About 60% of cardiac progenitor cells cultured in growth medium were positive for α-sarcomeric actin, although it was not organized in the cytoplasm as cytoskeleton (FIGS. 13A and 13B, left). Control cardiac progenitor cells cultured in growth medium were negative for α-smooth muscle actin and von Willebrand Factor staining. In contrast, clones of cardiac progenitor cells exposed to conditioned media for 4 days stained positively for α-sarcomeric actin, α-smooth muscle actin, and von Willebrand Factor (FIGS. 13A and 13B, right). Furthermore, some of the conditioned media-expanded cardiac progenitor cells that were positive for α-sarcomeric actin or α-smooth muscle actin possessed well-organized actin fiber structure that was not observed in cardiac progenitor cells cultured in growth medium. Cardiac progenitor cells expanded in conditioned media for 4 days no longer expressed c-kit. Thus, immunocytochemistry demonstrated that conditioned media promoted cardiac progenitor cell expansion and differentiation into 3 different cardiac cell lineages. Importantly however, the conditioned media did not appear to induce terminal cardiac myocyte differentiation of the cardiac progenitor cells as mature sarcomeric organization or spontaneous beating was never observed in these experiments.

### Example 11

### Conditioned Media Protects Cardiac Progenitor Cells Exposed to Hypoxia

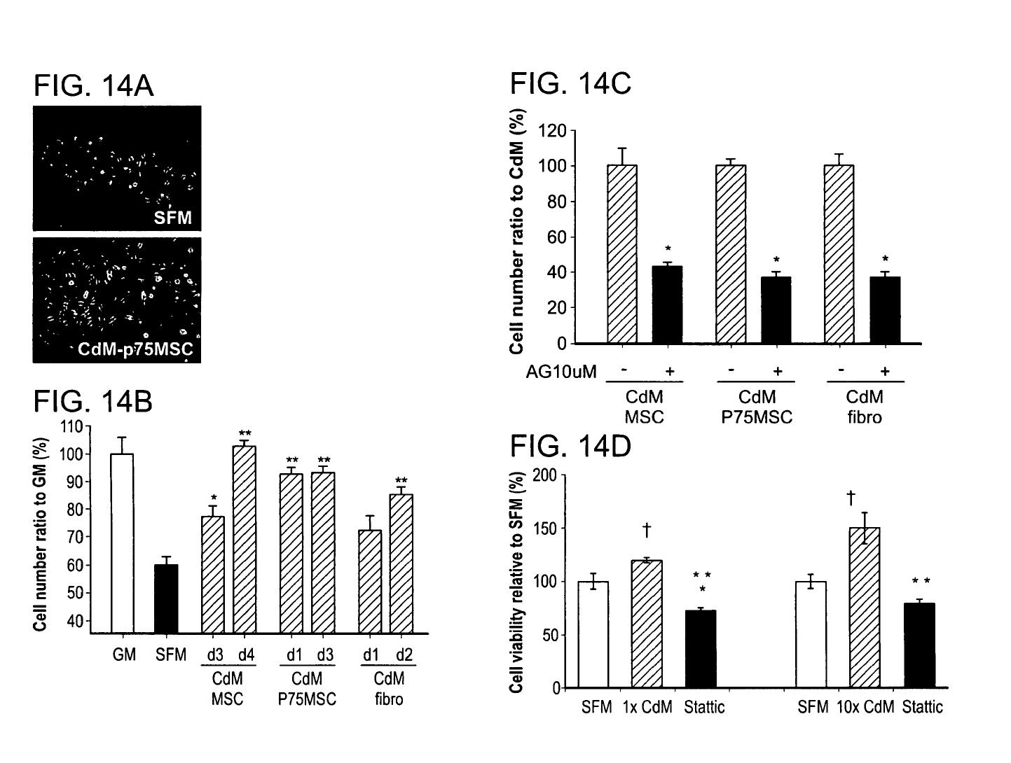

Because cardiac stem cells and cardiac progenitor cells in patients with myocardial infarction are likely to be exposed to ischemic conditions, it was determined whether conditioned media influenced the response of cardiac progenitor cells to hypoxic conditions for 48 hours. The conditioned media from MSCs and p75MSCs significantly promoted the survival of cardiac progenitor cells compared with serum-free medium (FIGS. 14A and 14B). As compared with the growth medium (positive control; 100±6.2%), survival of cardiac progenitor cells in serum-free medium was 59.9±3.2% while that of cardiac progenitor cells incubated in conditioned media from MSCs was 90.3±6.0% (P<0.05 vs serum-free medium) and from p75MSCs was 93±1.5% (P<0.0001 vs serum-free medium). Despite having a total protein content of about 15 fold less than that of the serum-containing cardiac progenitor cell growth medium (MSC and p75MSC Conditioned media, 0.11-0.13 mg/ml; growth medium, 1.92 mg/ml), cell survival in conditioned media from 3 of the 4 MSC donors tested was not significantly different from that in growth medium (MSC donor 4, p=0.66; p75MSC donor 1, p=0.33; p75MSC donor 3, p=0.37). Similar to its effects on cardiac progenitor cell proliferation, the Jak2/STAT3 pathway inhibitor AG490 also inhibited the protective effects of the conditioned media during hypoxia (FIG. 14C). In addition, the STAT3-specific inhibitor, Stattic, blocked the protective effects of 1× and 10×MSC Conditioned media (FIG. 14D), indicating that phosphorylation of STAT3 at Tyr⁷⁰⁵ in cardiac progenitor cells exposed to MSC conditioned media is responsible for its protective effects during hypoxia exposure.

### Example 12

### Factors Secreted by hMSCs

The protective effects of conditioned media from MSCs on cultured cardiomyocytes and endothelial cells under hypoxia has been demonstrated (Iso et al., Biochem Biophys Res Commun 2007; 354:700-706). In the previous study, human MSCs were found to express and secrete several cardioprotective factors. Conditioned media generated under serum-free conditions from p75MSCs also contained such factors: adrenomedullin, 3.12±0.37 ng/ml; hepatocyte growth factor, 0.36±0.17 ng/ml; LIF, 5.4±3.8 pg/ml; stromal-derived factor-1, 1.18±0.08 ng/ml; and vascular endothelial growth factor, 0.83±0.04 ng/ml (mean±SD). In addition to these growth factors/cytokines, MSCs and p75MSCs both secreted Dickkopf-1, an inhibitor of the Wnt signaling pathway (MSCs, 3.21±0.13 ng/ml; p75MSCs, 4.64±0.06 ng/ml; mean±SD). It has been shown that Wnt signal modulators play an important role in cardiac development and repair.

To attempt to identify the active secreted factors in MSC Conditioned media that affect cardiac progenitor cells an extensive series of antibody blocking experiments was performed to prevent the proliferation of cardiac progenitor cells when incubated in 1× or 10×MSC conditioned media that was previously incubated in antisera. Several proteins known to activate STAT3 by way of the gp130 receptor such as LIF, IL-6, IL-11, CNTF, and oncostatin M were blocked. Blocking none of these ligands reduced cardiac progenitor cell proliferation in MSC conditioned media. In addition, blockade of IGF1, IGF2, HGF, FGF2, FGF5, VEGF, PDGF, PLGF, CTGF, MCSF, GCSF, SDF1, TIMP1, TIMP2, gremlin, inhibin beta A, pleiotrophin, periostin, or leptin did not significantly reduce cardiac progenitor cell proliferation in MSC conditioned media. Therefore, an untested factor, an unidentified MSC-secreted factor, or the orchestration of low levels of several active factors is likely responsible for CPC activation and protection by MSC conditioned media.

Systemic administration of hMSCs into immunodeficient mice with myocardial infarction leads to improved cardiac function in the absence of long-term engraftment [8]. Surprisingly, MSC Conditioned media from the same donor protected both cultured murine cardiomyocytes and human umbilical vein endothelial cells from cell death during hypoxia. The favorable effects of the hMSCs on cardiac repair appeared to reflect the impact of transitory paracrine action or of secreted factors rather than engraftment, differentiation, or cell fusion. Gnecchi et al. (FASEBJ 2006; 20:661-669) reported that conditioned media from genetically-modified rat MSCs overexpressing Akt prevented cardiomyocyte death both ex vivo and in vivo after myocardial infarction. Angiogenesis/arteriogenesis in mice with hindlimb ischemia has also been shown to be stimulated by conditioned media from hMSCs (Kinnaird et al., Circ Res 2004; 94:678-685).

Thus far, MSCs have been shown to protect against ischemic injury through both direct prevention of cell death and through the stimulation of angiogenesis (Kinnaird et al., Circ Res 2004; 94:678-685; Noiseux et al., Mol Ther 2006; 14:840-850; Gnecchi et al., FASEBJ 2006; 20:661-669). The results presented here suggest that MSCs may also promote cardiac repair by their impact on endogenous cardiac progenitor cells. These results are consistent with the observation that hMSCs promoted the proliferation, migration and neurogenesis of endogenous neural stem cells following implantation of the hMSCs into the hippocampi of immunodeficient mice (Munoz et al., Proc Natl Acad Sci USA 2005; 102:18171-18176).

Because of the relatively small number of hMSCs that engrafted and survived in the brain, it was hypothesized that secreted cytokines/growth factors acting either directly on neural stem/progenitor cells or indirectly through the stimulation of astrocytes was responsible for the striking effects. Surprisingly, as reported herein, conditioned media from hMSCs promoted the proliferation of cardiac progenitor cells and protected them from the negative effects of hypoxia whereas the conditioned media did not propagate cardiac fibroblasts. These findings suggest that factors secreted by hMSCs may stimulate and protect endogenous cardiac stem/progenitor cells without increasing fibrosis in vivo, thereby promoting reparative myogenesis and angiogenesis/arteriogenesis in the heart after injury.

Insulin-like growth factor (IGF)-1 has been shown to have both mitogenic and anti-apoptotic effects on CSCs/cardiac progenitor cells (Urbanek et al., Circ Res 2005; 97:663-673). However, conditioned media that was generated under serum-free conditions from hMSCs contained undetectable levels of IGF-1 by ELISA and neutralization of IGF-1 with blocking antibodies did not alter cardiac progenitor cell growth in MSC conditioned media. It was also discovered that there were low levels of LIF and undetectable levels of EGF and bFGF in MSC conditioned media, and serum-free α-MEM supplemented with these factors alone did not induce cardiac progenitor cell proliferation. In addition, blocking antibodies against LIF and bFGF (FGF2) did not alter cardiac progenitor cell growth in MSC conditioned media. Thus, the effects of conditioned media is attributable to other factors contained in conditioned media from hMSCs.

Exposure to conditioned media from MSCs induced phosphorylation and nuclear localization of STAT3 in cardiac progenitor cells. STAT3 activation has previously been shown to influence various functions of stem/progenitor cells. It is essential for the self-renewal of mouse embryonic stem (ES) cells and has also been shown to play a role in the differentiation of mouse ES cells into beating cardiomyocytes (Foshay et al., Stem Cells 2005; 23:530-543). Transduction of the constitutively-activated form of STAT3 into HSCs increased the ability of the HSCs to rescue hematopoiesis in lethally-irradiated recipients (Chung et al., Blood 2006; 108:1208-1215). The proliferation and cell fate determination of neural precursors was also reported to be regulated by STAT3 activation (Yoshimatsu et al., Development 2006; 133:2553-2563; Gu et al., J Neurosci Res 2005; 81:163-171). The essential role of STAT3 in cardiac progenitor cells exposed to MSC Conditioned media was confirmed by the Jak2/STAT3 inhibitor (AG490) and the STAT3-specific inhibitor (Stattic) that prevented cardiac progenitor cell proliferation and survival. Although the PI3K/Akt pathway inhibitor LY294002 also reduced the cardiac progenitor cell growth induced by MSC conditioned media, the effect of LY294002 was less prominent than of AG490. However, the inhibitory effect of AG490 was not dominant to LY294002 on cardiac progenitor cell growth and survival in serum-free medium and the growth medium. These results indicate that STAT3 activation plays a crucial role in the cardiac progenitor cell growth and survival induced by secreted factors from hMSCs.

Because fibroblasts are well known to support the growth of various stem cells by their secretion of factors (Richards et al., Nat Biotechnol 2002; 20:933-936; Prowse et al., Proteomics 2005; 5:978-989; Kim et al., Cell 2005; 121:823-835; Messina et al., Circ Res 2004; 95:911-921) conditioned media from fibroblasts was used as positive control in the present study. Fibroblasts mediate tissue maintenance via paracrine action on other cell types (Manabe et al., Circ Res 2002; 91:1103-1113). Similar to MSCs, fibroblasts may also contribute to stem cell niches. Importantly, however, several in vivo studies have demonstrated that the administration of fibroblasts is less beneficial for tissue repair compared with stem/progenitor cells. Fibroblasts can induce fibrosis and influence tissue remodeling after injury. Accumulating evidence has shown that factors secreted by MSCs rather than by fibroblasts accelerate angiogenesis and wound healing (Miyahara et al., Nat Med 2006; 12:459-465; Hutcheson et al., Cell Transplant 2000; 9:359-368; Han et al., Plast Reconstr Surg 2006; 117:829-835; Han et al., Ann Plast Surg 2005; 55:414-419; Xu et al., Coron Artery Dis 2005; 16:245-255; Ninichuk et al., Kidney Int 2006; 70:121-129). Factors from MSCs also have an immunosuppressive property (Aggarwal et al., Blood 2005; 105:1815-1822). In addition to those effects, MSCs are multipotent and may contribute directly to cardiac and vascular cells, whereas fibroblasts lack multipotency. Thus, MSCs can promote tissue repair by a variety of mechanisms that are lacked by fibroblasts.

Cardiac stem cells/cardiac progenitor cells are involved in maintaining cardiac homeostasis during the course of life (Anversa et al., Circulation 2006; 113:1451-1463). Cardiac stem cells grow as cardio spheres. In contrast adherent cardiac progenitor cells are derived from cardiac stem cells. Although cardiac stem cells/cardiac progenitor cells are unable to completely regenerate cardiac tissue after injury, they represent a novel therapeutic target to enhance inherent cardiac regeneration. Factors secreted by hMSCs can activate and protect resident cardiac progenitor cells in culture and may act in a similar manner in vivo. Therefore, after injury, in addition to protecting cardiac cells, promoting angiogenesis/arteriogenesis, mediating matrix remodeling, and immunomodulation, infusion of hMSCs or standardized subpopulations such as p75MSCs may enhance endogenous tissue regeneration by activating and protecting CSC/Cardiac progenitor cells.

### Example 13

### Agents Present in Conditioned Media Preserve Cardiac Function

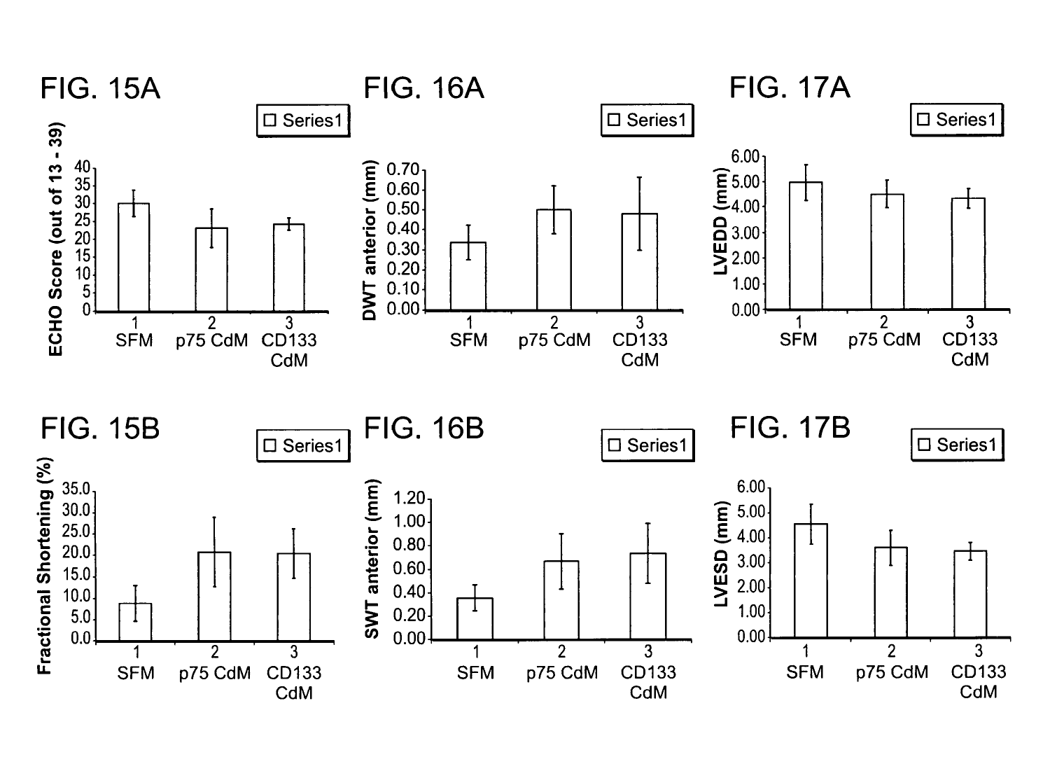

To induce cardiac ischemia, immunocompetant C57/bl6 mice (males, 8-10 weeks of age) underwent permanent ligation surgery. The mice were intubated and ventilated and the left anterior descending coronary artery (LAD) was ligated under microscopy using 7-O suture. The animals were recovered and returned to their cages. At 24 hours following the ligation, the animals received an intracardiac injection (left ventricle lumen, intra-arterial) of 200 ul of serum free medium (alpha MEM, vehicle) or 200 ul of 32× concentrated conditioned medium (CdM) from p75dMSCs or 200 ul of 32× concentrated medium (CdM) from CD133dMSCs. The vehicle or CdM was slowly infused over 1-2 minutes through a 30.5 gauge needle. Echocardiography was performed 1 week after the myocardial infarction. These methods preserved cardiac function in the treated animals. Following myocardial infarction, animals that received conditioned media showed markedly improved cardiac function relative to untreated control mice (FIGS. 15-17).

### Example 14

### Intracardiac Administration of CD133dMSCs CdM Reduced Cerebral Infarct Volumes in Mice

To evaluate the protective abilities of CD133dMSCs and their secreted factors, CD133dMSCs (cells) or concentrated CD133dMSC conditioned media (CdM) was administered to immunodeficient mice (males, 6-8 weeks old) 1 day after pMCAL. For comparison, concentrated CdM from p75dMSCs and hMSCs was administered. Each of the agents was infused slowly into the left ventricle of the heart in a 100 microliter volume (intra-arterial). The CdMs were generated from 90% confluent cells and were concentrated in a manner to normalize protein concentrations. By ANOVA, the protein concentrations of the individual CdMs did not differ significantly (p75dMSC CdM, 1.97±0.02 mg/ml; CD133dMSC CdM, 2.16±0.19 mg/ml, hMSC CdM, 2.06±0.06 mg/ml). Of note, whereas the CD133dMSC CdM and the hMSC CdM were both concentrated 40 fold to reach the protein determination values above, the p75dMSC CdM required 48.5 fold concentration to reach similar values. At 48 hours after treatment, the mice were euthanized and cortical infarct volumes were determined by cresyl violet staining. ANOVA with Bonferroni post hoc testing was used to determine differences in treatment effects.

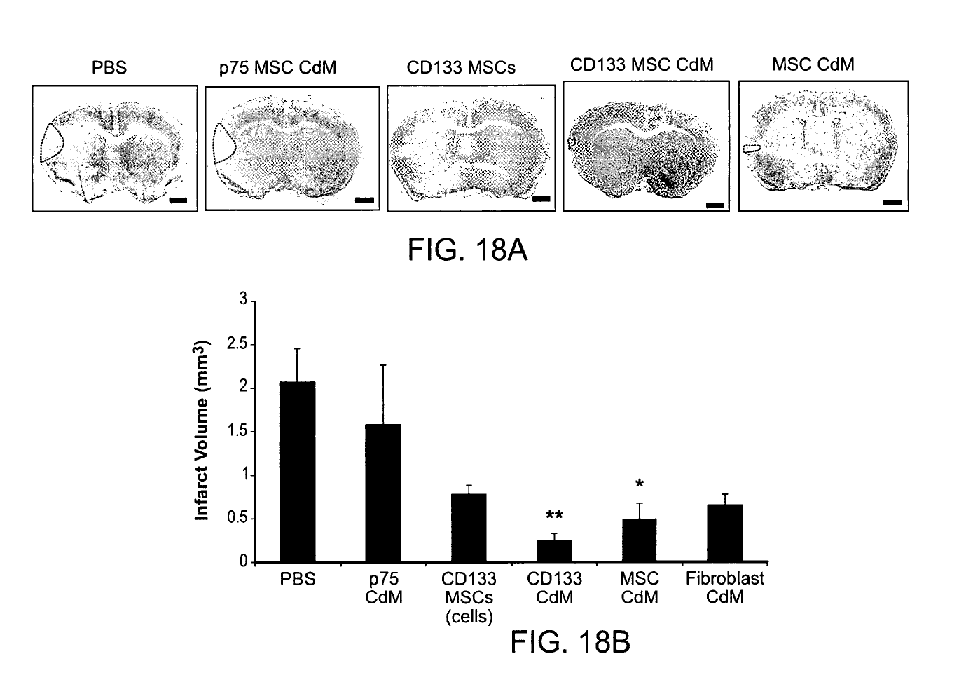

Infusion of p75dMSC CdM or CD133dMSCs (cells) did not significantly reduce cortical infarct volumes after stroke (FIGS. 18A and B). In contrast, CdM from typically-isolated hMSCs and CD133dMSC CdM both significantly reduced infarct volumes, with CD133dMSC CdM providing the greatest level of protection against cerebral ischemia (PBS, 2.1±0.86 mm³, n=5; MSC CdM, 0.49±0.40 mm³, n=5, p<0.05, CD133dMSC CdM, 0.25±0.15 mm³, n=6, p<0.01; (FIGS. 18A and B). The infusion of CD133dMSC CdM 1 d after pMCAL markedly limited the progression of ischemic injury so that the zone of infarction did not reach the typical size observed at day 3. These data indicate that the isolation of CD133-positive non-hematopoietic progenitor cells from human bone marrow enriches for a subpopulation of stem/progenitor cells with a secreted repertoire of factors that protect against stroke.

### Example 15

### Administration of CD133dMSC CdM Improves Motor Function After Stroke

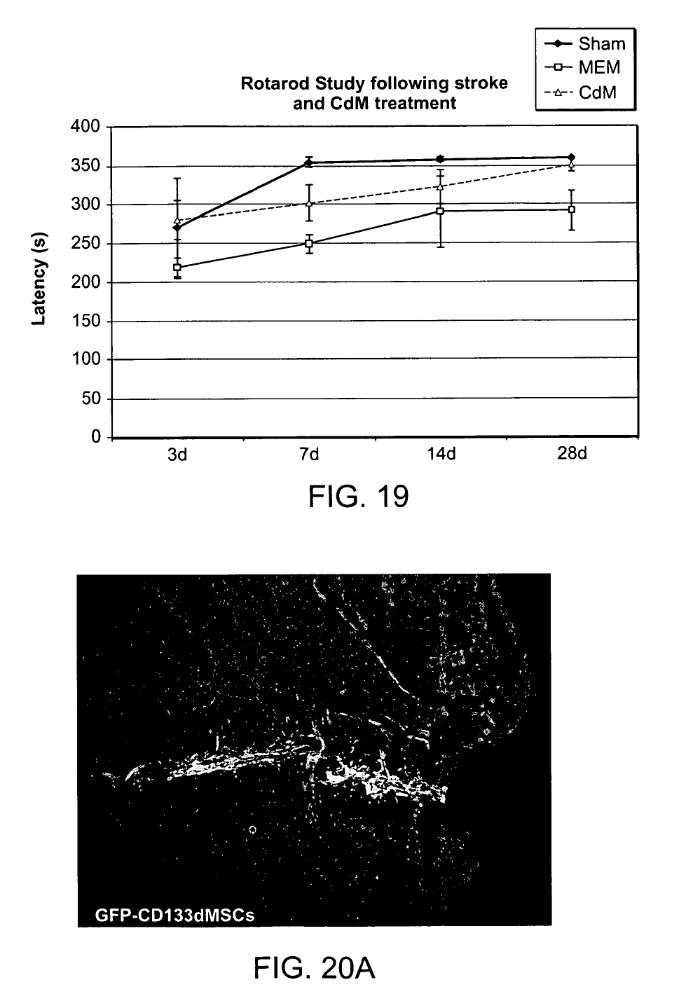

For studies in immunocompetent mice, the MCA was permanently ligated and delivered 200 microliters of 40× CdM from CD133dMSCs at 4 hours after the onset of ligation. Control animals received alpha MEM (MEM, vehicle) instead of CdM. Sham operated mice underwent the entire surgery but did not have the MCA ligated (suture passed underneath the MCA but not tied). Behavioral assessment of motor function was performed by rotorod testing at 3, 7, 14, and 28 days after stroke. At day 28, the mice that received CdM had significantly increased latency to fall times (better motor function) compared with those that received MEM, and were not significantly different than sham operated mice (FIG. 19, day 28, CdM vs. MEM; p<0.01).

### Example 16

### CD133dMSCs Express mRNAs of Protective Secreted Factors Following Transplantation into Hypoxic/Ischemic Cerebral Tissue

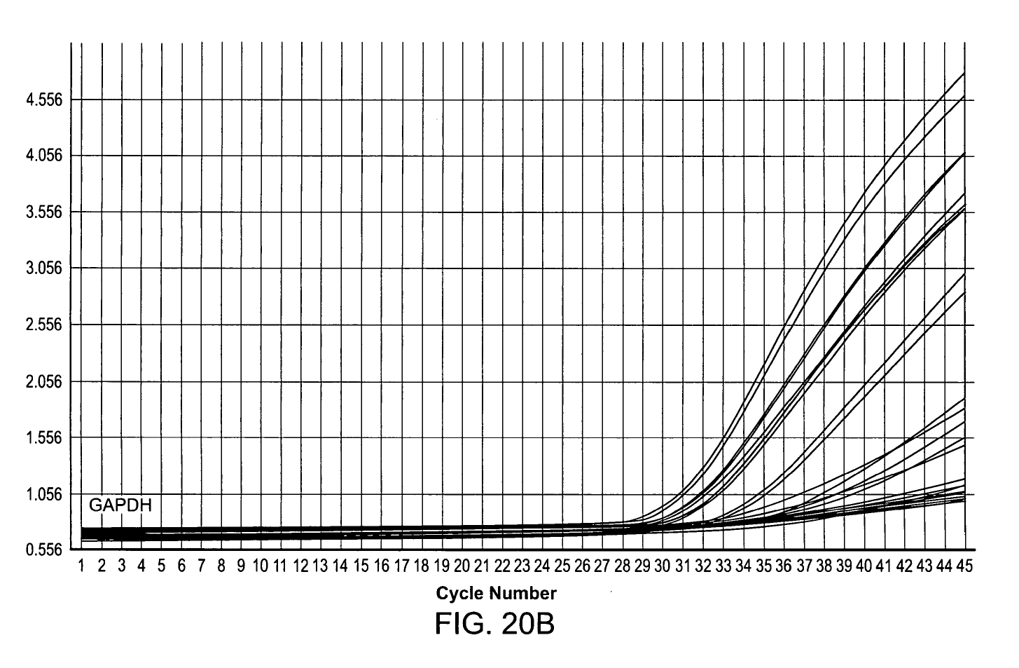

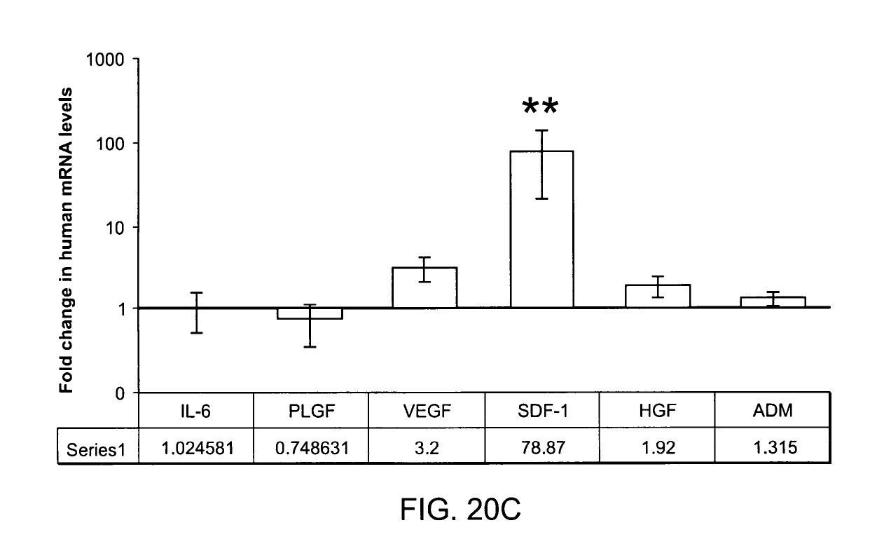

To examine whether CD133dMSCs express mRNAs for protective secreted factors while located in injured cerebral tissue 100,000 lentivirally GFP-tagged CD133dMSCs were injected directly into the brains of immunocompetent mice 1 day after pMCAL surgery or sham surgery. Mice were euthanized forty-eight hours later. The mouse brains were cut on a polyacrylic brain block and total RNA was isolated from the upper quadrant of the brain that contained the infarct volume and the injected CD133dMSCs. Epifluorescent microscopy was used to locate the GFP-CD133MSCs 48 hrs post injection (FIG. 20A). Assays with human-specific real time RT-PCR detected human mRNA transcripts for GAPDH, IL6, PLGF, VEGF, SDF1, HGF, and adrenomedullin (ADM) (FIGS. 20B and 20C). For several mRNAs of secreted proteins, the levels of detected human mRNAs increased in brains with pMCAL compared with sham-operated brains that received the same cell injection and surgery but did not have the MCA ligated (FIG. 20C).

### Example 17

### Selection and Expansion of CD133dMSCs after Transduction with Lentiviral shRNA Vectors to Knockdown the Expression and Secretion of SDF-1

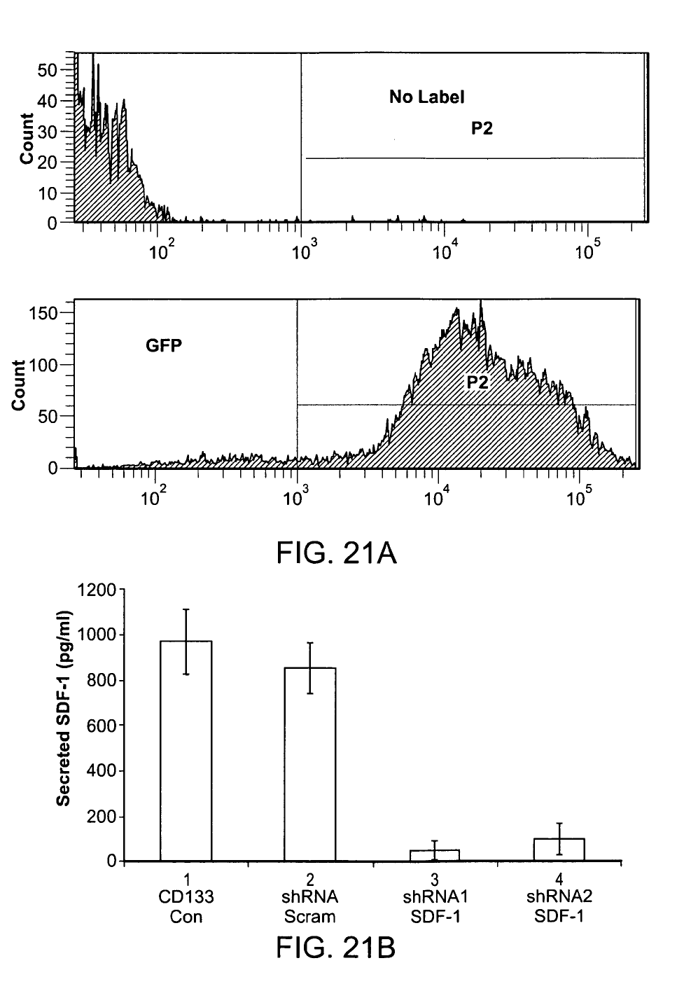

Based on observations that CD133dMSCs increased their secretion of SDF-1 in culture following exposure to hypoxia and mRNA expression in vivo after injection into stroke penumbra, the role of SDF-1 in mediating the benefits of CD133dMSC CdM was explored using lentiviral shRNA knockdown of SDF-1 with puromycin-selectable vectors. In preliminary studies, kill curves were performed with transduced CD133dMSCs incubated in puromycin-containing culture medium to remove cells that were not transduced by lentivirus. 2 μg/ml puromycin was found to be sufficient to remove all untransduced CD133dMSCs after 3 days. Following lentiviral transduction of expanded CD133dMSCs from a single donor with a scrambled shRNA vector, 2 different shRNAs vectors against SDF-1, or a control selectable GFP vector, ELISAs and FACS assays were performed to characterize the cells. After 2 weeks of expansion in puromycin-containing medium, 100% of CD133dMSCs were found to be GFP positive following transduction with the GFP control vector and puromycin selection (see FACS histograms, FIG. 21A). ELISAs of 48 hr CD133dMSC-conditioned medium demonstrated a dramatic knockdown of SDF1 secretion in CD133dMSCs that had been transduced with SDF1-specific shRNAs compared with cells transduced with a scrambled shRNA containing vector (SDF-1 shRNA 1, 94% knockdown; SDF-1 shRNA2, 88% knockdown; FIG. 21B). SDF1 secretion did not differ between the original CD133dMSCs (control, untransduced) and those that were transduced with the scrambled shRNA vector (FIG. 21B).

### Example 18

### CD133dMSC-Conditioned Medium (CdM) Rescues Mouse Neural Stem/Progenitor Cells During Growth Factor Withdrawal and Hypoxia/Ischemia Exposure, in Part Through SDF-1

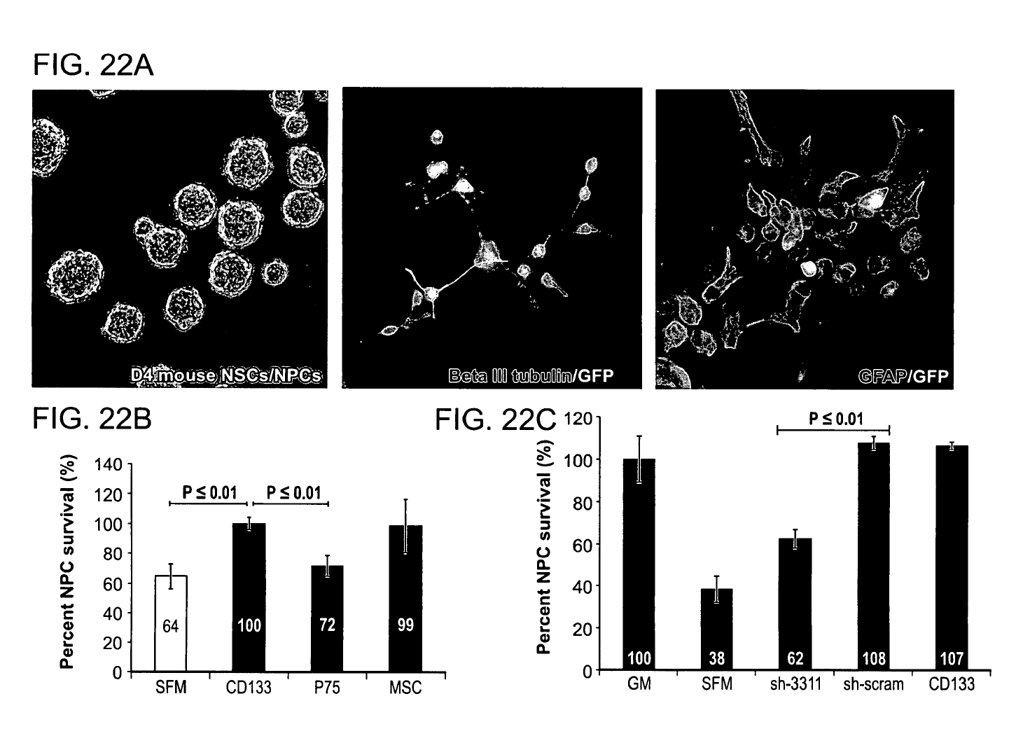

Neural stem/progenitor cells (NSCs/NPCs) were isolated from GFP transgenic mice in order to examine the ability of secreted factors from CD133dMSCs to protect neural stem/progenitor cells during growth factor withdrawal and hypoxia/ischemia exposure. The NPCs readily differentiated into immature beta III tubulin-positive neurons and GFAP-positive astrocytes in the appropriate differentiation mediums (FIG. 22A). For neural progenitor cell protection assays, serum-free low glucose alpha MEM (SFM) and hypoxia exposure (1% oxygen) was used to simulate ischemic conditions for mNPCs. Neurosphere cultures were dissociated into single cell suspensions and the cells were plated onto laminin/poly D lysine-coated cell ware in NSC/NPC growth medium containing EGF, bFGF, Heparin and B27. After 2 days of adherent growth, the growth medium was switched to serum-free alpha MEM (SFM) or serum free 1× CdM from CD133dMSCs, p75dMSCs, or hMSCs for 48 hrs. CD133dMSC CdM provided significant protection against growth factor/nutrient withdrawal-induced cell death compared with SFM (P≦0.01, FIG. 22B). The level of NPC protection provided by CD133dMSC CdM did not differ from that conferred by hMSC CdM. However, CD133dMSC CdM protected significantly greater numbers of NPCs when compared with the protection provided by p75dMSC CdM (P ≦0.01, FIG. 22B). These results indicated that CD133dMSC CdM contained different types or levels of secreted factors that benefited NPC survival compared with p75dMSC CdM. Notably, CdM from CD133dMSCs and hMSCs both protected as well as NPC/NSC growth medium, despite lacking appreciable amounts of EGF and bFGF (<2 pg/ml), implying that other factors or combinations of factors secreted by CD133dMSCs were responsible for protecting the NPCs. To evaluate the role of secreted SDF-1 in mediating the protective effects of CD133dMSC CdM on NPCs, the growth factor withdrawal experiment was performed under hypoxic (1% oxygen) conditions and the protection conferred by CdM was compared with unmanipulated CD133dMSCs, those lentivirally transduced with the scrambled shRNA vector, and those transduced with the SDF-1 shRNA vector (94% SDF-1 knockdown by ELISA). Knockdown of SDF-1 in CD133dMSC CdM reduced is capacity by over 50%, indicating that it is a key factor secreted by CD133dMSCs that protects NPCs from ischemic injury (P≦0.01 compared with Scram, FIG. 22C). These data support the hypothesis that administration of CD133dMSC CdM after stroke may lead to improved repair by increasing the survival of endogenous NSCs/NPCs.

The results described in the Examples were obtained using the following methods and materials.

Isolation and Preparation of hMSCs, CD133dMSCs, and p75dMSCs

MSCs were isolated from bone marrow aspirates, expanded, and banked as frozen vials of cells (Tulane Center for the Preparation and Distribution of Adult Stem Cells www.som.tulane.edu/gene_therapy/distribute.shtml). Briefly, 2-10 cc iliac crest aspirates were obtained from healthy human donors. Mononuclear cell fractions were obtained by discontinuous ficoll density gradient centrifugation and extraction of the buffy coat (Ficoll-Paque PLUS, GE Healthcare, Piscataway, N.J.). All cells were cultured in nunclon delta-coated 15 cm² dishes (Nunc, Thermo Fisher Scientific, Rochester, N.Y.). For the isolation of typical plastic adherent hMSCs, the mononuclear cell fraction was cultured directly in Complete Culture Medium (CCM) containing alpha MEM (Invitrogen, Carsbad, Calif.), 20% fetal bovine serum (lot selected for rapid growth of hMSCs, Atlanta Biologicals, Lawrenceville, Ga.), 100 units/ml penicillin, 100 μg/ml streptomycin, and 2 mM L-glutamine (Mediatech Inc., Hendron, Va.). To isolate the CD133dMSCs and p75dMSCs from total bone marrow mononuclear cells, MACS was performed using antibodies conjugated to dextran-coated iron beads according to the manufacturer's instructions (CD133 microbeads, CD271 microbeads [p75LNGFR]; Miltenyi Biotech, Auburn, Calif.).

### Phenotypic Analysis by Flow Cytometry

Pellets of 10⁵ to 0.5×10⁶ cells were suspended in 0.5 ml PBS and were incubated for 30 minutes at 4° C. with monoclonal mouse anti-human antibodies that were pre-titered for flow cytometry. All antibodies except those against CD133 (Miltenyi Biotech) and CD105 and NG2 (Beckman Coulter, Miami, Fla.) were purchased from BD Biosciences Pharmingen (San Diego, Calif.). After labeling, the cells were washed twice with phosphate buffered saline (PBS) and analyzed by closed-stream flow cytometry (Epics XL, Beckman Coulter; LSR II, Becton Dickinson, Franklin Lakes, N.J.).

### Microarray Assays

For microarray assays of expressed genes, freshly isolated bone marrow mononuclear cells from different aspirate donors were sorted for CD133-positive cells or p75LNGFR-positive cells (MACS) for RNA isolation (High Pure RNA Isolation Kit, Roche Applied Science, Indianapolis, Ind.). To obtain enough material from the fresh cells for microarray assays, ileac crest aspirates from each side (left/right) of a given donor were sorted and the cells were lysed and combined. For cultured cells, P1 hMSCs, CD133dMSCs, and p75dMSCs (n=2 donors per cell type) were seeded in 15 cm² dishes in CCM at 100 cells/cm², incubated until they reached 60 to 70% confluency (5-7 days), and lifted with trypsin/EDTA for RNA isolation (P2). Microarray methods including sample preparation, analysis by dChip (Li C and Wong, Proc Natl Acad Sci USA 2001; 98:31), hierarchical clustering, and analyses for gene ontologies are provided below.

Proliferation Assays of hMSCs, CD133dMSCs, and p75dMSCs

Passage 3 hMSCs, CD133dMSCs, and p75dMSCs (n=3 per cell type) were expanded in CCM, lifted, and plated at 100 cells/cm² in 6 well plates (Nunclon, Nunc, Thermo Fisher Scientific, Rochester, N.Y.). Cells were grown in CCM under normoxic or hypoxic (1% oxygen) conditions for 2, 4, or 8 days prior to sampling (Thermo Electron Corporation incubator model 3130, Houston, Tex.). At each time point, cells were lifted with trypsin/EDTA (Mediatek, Inc., Hendron, Va.), pelleted, and frozen at −80° C. For cells of each donor, 2 wells of the 6 well plate were combined and considered as a replicate (n=3 per plate, per timepoint). Cell numbers were quantified by dye-labeling of nucleic acids (CyQUANT, Invitrogen, Carlsbad, Calif.) in triplicate using a fluorescence plate reader (Biotek Synergy HT, BioTek Instruments, Inc., Winooski, Vt.).

### Differentiation Assays

Confluent cultures were prepared by plating CD133dMSC and p75dMSC P1 cells at 1,000 cells/cm² and incubating for 5 days in CCM. The cultures were then transferred to either osteogenic media or adipogenic medium. For chondrogenic differentiation, cells were harvested with trypsin/EDTA and micromass pellet cultures were prepared by centrifugation of 200,000 cells at 1000×g for 8 min in 15 ml conical tubes. Pellets were cultured at 37° C. with 5% CO₂ in 500 μl chondrogenic media. Detailed methods for differentiation assays are below.

### Production of Conditioned Mediums

Three donors each of hMSCs, CD133dMSCs, and p75dMSCs were used to produce conditioned mediums (CdM). Cells were seeded from frozen cryotubes containing approximately 1 million cells at passages 2 or 3, and then expanded, split, and utilized for CdM collection at passages 4 or 5. Cells were passaged 2 times with media changes every 3-4 days until 50% and 90% confluence was achieved for each donor. At these densities, the cells were washed twice with PBS and the medium was switched to serum-free alpha MEM (SFM). At the time of SFM application, duplicate plates from each donor at both densities were placed in incubators set to 37° C., 5% CO₂, and normal atmospheric oxygen (21%) or 1% oxygen. After 48 hours of incubation, the CdM was collected, filtered (0.2 μm PES membrane, Nalgene MF75, Rochester, N.Y.), and frozen at −80° C. The cells at the time of CdM collection were lifted with trypsin/EDTA, pelleted, and frozen (−80° C.). Cell numbers were quantified by dye-labeling of nucleic acids as above. For in vivo studies, CD133dMSC CdM was concentrated to 40-fold with a Labscale™ TFF diafiltration system using filters with a 5 kD cut-off (Millipore, Bedford, Mass.). Therefore only medium components above 5 kD were concentrated (base medium components and salts remained at 1×).

### ELISAs of Secreted Growth Factors and Cytokines

Sandwich enzyme linked immunosorbant assays (ELISAs) were performed to quantify the levels of selected growth factors and cytokines secreted by hMSCs, CD133dMSCs, and p75dMSCs. Detailed ELISA methods are provided below.

### MCA Ligation

Male immunodeficient (NOD/SCID beta 2 microglobulin^{-/-}) mice at 6-8 weeks of age were anesthetized with isoflurane (1-5%, to effect), and body temperature was maintained by keeping the animals on a heating pad. Under low-power magnification, the left temporal-parietal region of the head was shaved and an incision was made between the left orbit and left ear in the shape of a “U”. The parotid gland and surrounding soft tissue was reflected downward and an incision was made superiorly on the upper margin of the temporal muscle forward. The MCA was then visualized through the semi-translucent skull. A small burr hole (1-2 mm) was drilled into the outer surface of the skull just over the MCA. The skull was removed with fine forceps, and the dura was opened with a cruciate incision. The MCA was encircled with 10-0 monofilament nylon using a curved surgical needle and ligated (Henry Schein, Melville, N.Y.). In each animal, cessation of flow through the artery was verified visually. In addition, to ensure that the MCA had been ligated, a 27.5 gauge needle was used to break the vessel close to the suture (superior to the ligation). The small flap of facial skin was closed with Vetbond (3M, St. Paul, Minn.). Animal survival after the ligation surgery was 93%. One day after the MCA ligation, groups of mice were re-anesthetized and received a single injection of either 100 μl of PBS, 100 μl of PBS containing 1×10⁶ CD133dMSCs, or 100 μl of 40×CD133dMSC CdM (from the same donor) into the left ventricle lumen (intracardiac, arterial) using a 27.5 gauge needle. The presence of the needle in the left ventricle lumen was confirmed by draw-back of blood. Infusions were made slowly over 1 minute. All mice were euthanized 48 hrs after treatment for analysis.

### Microarray Sample Preparation

Samples for microarrays were prepared according to the manufacturer's directions. In brief, 8 μg of total RNA was used to synthesize double-stranded cDNA using commercially available reagents (Superscript Choice System/GIBCO BRL Life Technologies). After synthesis, the double stranded cDNA was purified by phenol/chloroform extraction (Phase Lock Gel, Eppendorf Scientific) and concentrated by ethanol precipitation. In vitro transcription was used to produce biotin-labeled cRNA (BioArray HighYield RNA Transcription Labeling Kit; Enzo Diagnostics). The biotinylated cRNA was then cleaned (RNAeasy Mini Kit; Qiagen), fragmented, and hybridized on the HG-U133 Plus 2.0 microarray chips (Affymetrix). These chips consist of over 54,000 oligonucleotides, representing over 31,000 human genes. After washing, individual microarray chips were stained with streptavidin-phycoerythrin (Molecular Probes), amplified with biotinylated anti-streptavidin (Vector Laboratories), stained again with streptavidin-phycoerythrin, and scanned for fluorescence (GeneChip Scanner 3000, Affymetrix) using the GeneChip Operating software 1.0 (GCOS, Affymetrix).

### Microarray Data Processing

GCOS recorded intensities for perfect match (PM) and mismatch (MM) oligonucleotides, and determined whether genes were present (P), marginal (M) or absent (A). The scanned images were then transferred to the dChip program (dChip reference). To allow comparisons between different microarrays, an array was chosen as the baseline array (CD133dMSC d5028, median intensity of 98) against which the other arrays were normalized at the probe intensity level. The dChip program then calculated the model based expression values using the PMs and MMs. Negative values were assigned a value of one.

Hierarchical Clustering Algorithm in dChip

The dChip program standardized the expression values for each gene by linearly adjusting their values across all samples to a mean of zero with a standard deviation of one. Individual genes were then clustered using an algorithm in dChip program that determined the correlation coefficients (r values) for the normalized expression values (distances between genes were defined as 1−r). Genes with the shortest distances between them were merged into super-genes, connected in a dendogram by branches with lengths proportional to their genetic distances, and then merged (centroid-linkage). This process was repeated n−1 times until all genes had been clustered. A similar algorithm was also used to cluster the samples. These standardization and clustering methods follow Golub et al. 1999; 286:531-537 and Eisen et al., Proc Natl Acad Sci USA 1998; 95: 14863-14868.

### Sample Clustering

All the sample clusterings were performed with the algorithm described previously using eight samples. Sample clusters were generated using the lists of genes obtained with:
(1) no filtering (all 54,675 transcripts)
(2) filtering for largest changes using coefficient of variation (CV, standard deviation of expression value for each gene divided by the mean expression value for that gene across all the samples) larger than 0.7 and a present call of at least 25% in the eight samples (5,517 transcripts)
(3) no filtering (54,675 transcripts), but averaging the signals from biological replicates (same condition but a different donor)
(4) filtering for largest changes using coefficient of variation larger than 0.7 and a present call of at least 25% in the five conditions (6,283 transcripts)

### Heat-Maps

A heat-map was generated using the algorithm described previously. The heat-map was generated using the same samples and genes as in (4) of the sample clustering.

### Gene Ontology

Based on the clustering, nine patterns of gene expression were identified in the similar level of hierarchy. The genes in these patterns were studied for GeneOntology (GO) terms, which provide information on the cellular component, biological process and molecular function of the protein product of the gene. Redundant probesets (based on Gene ID) were removed and p-values were calculated for each term using an exact hyper-geometric distribution, in the dChip program, to compare the frequencies of individual terms within the pattern to the frequencies of those terms on the entire microarray. P-values under 0.01 were considered significant. Gene ontology is described, for example, by Ashburner et al. (Gene ontology: tool for the unification of biology. The Gene Ontology Consortium. Nat Genet 2000; 25:25-9).

### Differentiation Assays

Confluent cultures were prepared by plating CD133dMSC and p75dMSC P1 cells at 1,000 cells/cm² and incubating for 5 days in CCM. The cultures were then transferred to either osteogenic media or adipogenic medium. The osteogenic medium consisted of alpha MEM containing 10% FCS, 1 nM dexamethasone, 0.2 mM ascorbic acid, and 10 mM β-glycerol phosphate (Sigma, St. Louis, Mo.). After incubation in osteogenic medium for 3 weeks with changes of medium every 3 or 4 days, the cells were washed in PBS, fixed in 10% neutral-buffered formalin for 20 minutes, and stained with 0.5% Alizarin Red (pH 4.1; Sigma) for 20 minutes before washing for three times for 5 minutes each with PBS. The adipogenic medium consisted of alpha MEM containing 10% FCS, 0.5 μM hydrocortisone, 0.5 mM isobutylmethylxanthine, and 60 μM indomethacin (Sigma). After incubation in adipogenic medium for 3 wk with media changes every 3 to 4 days, the cultures were washed, fixed, and stained with Oil Red-0 (Sigma). The Oil Red-0 solution was prepared by diluting 3 parts of 0.5% v/v stain in isopropanol with 2 parts water and clarified by filtration through a 0.2 μm filter. The cultures were incubated with the stain for 30 minutes before washing three times with PBS. For chondrogenic differentiation, cells were harvested with trypsin/EDTA and micromass pellet cultures were prepared by centrifugation of 200,000 cells at 1000×g for 8 minutes in 15 ml conical tubes. Pellets were cultured at 37° C. with 5% CO₂ in 500 μl chondrogenic media that consisted of high-glucose DMEM (Invitrogen) supplemented with 500 ng/ml BMP-6 (R & D Systems; Minneapolis, Minn.), 10 ng/ml TGF-(33 (Sigma), 0.1 μM dexamethasone, 50 μg/ml ascorbate-2 phosphate, 40 μg/ml proline, 100 μg/ml pyruvate, and 50 mg/ml ITS+Premix (6.25 μg/ml insulin, 6.25 μg/ml transferrin, 6.25 ng/ml selenious acid, 1.25 mg/ml BSA, and 5.35 mg/ml linoleic acid; Becton Dickinson). Pellets were fixed and embedded in paraffin, cut into 5 μm sections, and stained with Toluidine Blue sodium borate.

### ELISAs of Secreted Growth Factors and Cytokines

Sandwich enzyme linked immunosorbant assays (ELISAs) were used to quantify the levels of selected growth factors and cytokines secreted hMSCs, CD133dMSCs, and p75dMSCs. Cell cultures from 3 different donors were assayed under several different conditions: 1) cell density, 50% or 90% confluence; and 2) oxygen levels, normoxic (21% oxygen) or hypoxic (1% oxygen) conditions for 48 hours. ELISAs were performed according to the manufacturer's instructions (HGF, PLGF, VEGF, BDNF, DKK-1, PDGF-AB, EGF, β-NGF, and IGF-1: DuoSet ELISA Development System, R and D Systems, Inc., Minneapolis, Minn.; LIF: Quantikine protocol, R and D Systems, Inc.); NGF: Eₘₐₓ immunoassay system, Promega Corp., Madison, Wis.: Adrenomedullin: Enzyme Immunoassay Kit, Phoenix Pharmaceutical, Inc., Burlingame, Calif.). Basic-FGF was assayed with capture antibody (1:750, Sigma anti bovine/human bFGF CLONE FB-8 and # F6162) and Biotinylated anti-bFGF (0.25 μg/mL, Abcam polyclonal Ab12476; Abcam, Cambridge, Mass.). Streptavidin-HRP (R and D Rystems, Inc.) was used in all assays for biotinylated antibody detection. ABTS Enhancer (2,2′Azino-bis[3-ethylbenzothiazoline-6-sulfonic acid]) was used for substrate detection (Thermo Fisher Scientific, Rochester, N.Y.). Absorbance measurements for all samples and standards were performed in triplicate at 590 nm wavelength (Biotek Synergy HT). Standard curves of known protein concentrations were generated for each ELISA and linear line equations were used to determine protein concentrations in CdM samples.

The results described in Examples 7-10 were obtained using the following methods and materials.

### Preparation of Human Bone Marrow MSCs.

Human MSCs (hMSCs) and dermal fibroblasts were provided by the Tulane Center for the Preparation and Distribution of Adult Stem Cells (http://www.som.tulane.edu/gene_therapy/distribute.shtml) and prepared with protocols approved by an Institutional Review Board. In this study, hMSCs isolated by plastic adherence were defined as MSCs and the subpopulation derived from bone marrow cells positive for p75LNGFR were defined as p75MSCs.

To obtain MSCs, bone marrow aspirates were taken from the iliac crest of healthy adult donors. Mononuclear cells were isolated with the use of density gradient centrifugation (Ficoll-Paque, Amersham Pharmacia Biotech) and resuspended in complete culture medium consisting of α-MEM (GIBCO/BRL, Grand Island, N.Y.); 17% FBS (Atlanta Biologicals, Norcross, Ga.); 100 units/ml penicillin (GIBCO/BRL); 100 μg/ml streptomycin (GIBCO/BRL); and 2 mM L-glutamine (GIBCO/BRL). Cells were plated in 20 ml of medium in a 150 cm² culture dish and incubated in a humidified incubator (Thermo Electron, Form a Series II, Waltham, Mass.) with 95% air and 5% CO₂ at 37° C. After 24 h, nonadherent cells were removed. Adherent cells were washed twice with PBS and incubated with fresh medium. The primary adherent cells were cultured and propagated.

To obtain p75MSCs, bone marrow stem/progenitor cells were isolated by MACS using antibodies against the p75LNGFR. Freshly isolated bone marrow mononuclear cells from the Ficoll gradient were resuspended in 0.4 ml of PBS containing 0.5% bovine serum albumin and 2 mM EDTA. After adding mouse anti-human p75LNGFR antibody conjugated to magnetic beads (CD271, Miltenyi Biotech, Auburn, Calif.), the sample was incubated for 30 min at 4° C.; and then applied to a magnetic column (LS Column; Miltenyi Biotech). The bound fraction was eluted with 5 ml of MACS buffer and the cells were concentrated by centrifugation at 1000×g for 8 min. After resuspension, the entire isolate was cultured in complete culture medium. MSC-like cells appeared as small colonies after about 1 week, and the cells were expanded.

Characterization of p75MSCs.

To characterize the surface antigens of p75MSCs, cells were analyzed with the use of a fluorescence-activated cell sorting (FACS) (FACScan flow cytometer, Becton Dickinson, Franklin Lakes, N.J.). Cells were incubated with fluorescein isothiocyanate (FITC)-conjugated mouse monoclonal antibodies against human CD31, CD34, CD44, CD45, CD90, and CD105 (all from Becton Dickinson). Isotype-specific antibodies served as controls.

Characterization of differentiation was performed as described previously (Munoz et al., Proc Natl Acad Sci USA 2005; 102:18171-18176). The p75MSCs were grown to 80% confluence in complete culture medium. For osteogenic differentiation the medium was changed to α-MEM containing 10% FCS and was supplemented with 50 μM ascorbic acid 2-phosphate, 1 nM dexamethasone, and 20 mM β-glycerophosphate. For adipogenic differentiation the medium was changed to α-MEM containing 10% FCS and was supplemented with 0.5 μM dexamethasone, 0.5 μM isobutylmethylxanthine, and 50 μM indomethacin (Prockop et al., Science 1997; 276:71-74; Scadden, Nature 2006; 441:1075-1079). The medium was replaced every 3-4 days for 21 days. Cells were fixed and stained with Alizarin red S (pH 4.1, Sigma, St. Louis, Mo.) and Oil red O (Fisher Scientific, Liberty Lane Hampton, N.H.).

### Isolation and Culture of Cardiac Stem Cells.

Adult CSCs were isolated from the ventricles of Fischer 344 rats as described previously (Prockop et al., Science 1997; 276:71-74; Scadden, Nature 2006; 441:1075-1079). Cells from a single clone were obtained by the sorting and propagation of single cell-derived clones that were infected with a retrovirus carrying enhanced green fluorescent protein (EGFP). The viral titer was 10⁶ cfu/ml. Cells from a single clone that were positive for c-kit and that expressed EGFP were used for experiments. CSCs derived from the clone were cultured in a modified neural stem cell medium (mNSCM) consisting of DMEM/F12 (ratio 1:1) (GIBCO/BRL) supplemented with insulin-transferrin-selenite, 10 ng/ml basic fibroblast growth factor (bFGF), 20 ng/ml epithelial growth factor (EGF), and 10 ng/ml leukemia inhibitory factor (LIF) as described previously (Prockop et al., Science 1997; 276:71-74).

To grow adherent CPCs from spheroid CSCs that were previously grown in mNSCM, CSCs were plated at 500 cells/cm² and cultured in mNSCM supplemented with 2% FBS (growth medium).

### Isolation and Culture of Adult Rat Cardiac Fibroblasts.

Procedures conformed to the Guide for the Care and Use of Laboratory Animals published by the US National Institute of Health. The animal protocol for this study was approved by the Institutional Animal Care and Use Committee of University of Vermont. Ventricular fibroblasts were isolated from adult Sprague-Dawley rats. The hearts were minced and enzymatically dissociated into single cell suspension. Nonmyocytes were separated by the discontinuous density gradient centrifugation and cultured in DMEM/F-12 supplemented with 10% FBS. Second passage of the cells was used for experiments.

### Preparation of Serum-Free Conditioned Medium (CdM).

Passage 4 to 8 MSCs, p75MSCs or fibroblasts were used to generate CdM. To prepare conditioned media, the cells were cultured to 80 to 90% confluence in 150 cm² dishes with complete culture medium. These cells were washed with PBS (2 times) and incubated with 20 mls of fresh serum-free α-MEM in standard conditions without any supplements or growth factors for 48 hrs. The culture medium was then collected, filtered (0.22 μm filter), and stored at −80 C.°. For some experiments, conditioned media was concentrated up to 10-fold with the use of a Labscale™ TFF diafiltration system (Millipore, Bedford, Mass.).

### Cell Culture in Conditioned Media and Evaluation of Cell Number.

Serum-free conditioned media was prepared as described previously (Kiel et al., Cell 2005; 121:1109-1121). cardiac progenitor cells and cardiac fibroblasts were plated at 500 cells/cm² and cultured in their growth medium. Three days after plating the medium was removed, the wells were washed twice with PBS, and the cells were then exposed to conditioned media or to fresh serum-free medium (α-MEM). For time course proliferation studies, the conditioned media and serum-free medium were changed every 2 days. In the signal transduction inhibitor studies the following pharmacological inhibitors, were used (10 micromolar): AG490, an inhibitor of Jak2/STAT3 pathway; Stattic, a specific inhibitor of STAT3 phosphorylation at Tyr⁷⁰⁵; LY294002, inhibitor of phosphatidylinositol 3-kinase (PI3K)/Akt pathway; and PD98059, extracellular signal-regulated kinase (ERK) inhibitor. All inhibitors were from Calbiochem (EMD Chemicals, San Diego, Calif.) and were dissolved in dimethyl sulfoxide (DMSO). Cardiac progenitor cells were cultured in conditioned media with the inhibitors or with the equivalent volume of DMSO as a control for 48 hrs. In the protection study, 3 days after plating, the medium was replaced with either the conditioned media or serum-free medium and the cells were exposed to hypoxia in a specialized incubator (1% oxygen) for 48 hours. The hypoxia incubator was a model that measured both CO₂ and O₂ (Thermo Electron, Form a Series II, model 3130). Oxygen was maintained at 1% by the injection of nitrogen gas and was monitored continuously.

Cell numbers were quantified by the fluorescent labeling of nucleic acids (CyQuant dye; Molecular Probes, Carlsbad, Calif.) and with a microplate fluorescence reader (FL_{X}800; Bio-Tek Instruments Inc., Winooski, Vt.) set to 480 nm excitation and 520 nm emission. Each experiment was repeated a minimum of 3 times.

### Immunocytochemistry.

Cardiac progenitor cells were fixed with 4% paraformaldehyde in 1×PBS. Non-specific binding was limited by a 1 hour incubation in PBS containing 5% goat serum and 0.4% triton X-100. Primary antibodies were applied to the sections and were incubated overnight at 4° C. After washing 3×5 min with PBS, secondary antibody that was diluted 1:1000 (Alexa 594, Molecular Probes) was applied to the slides for 1 hour at room temperature (RT). After 3×5 minute washes, the slides were mounted with Vectashield containing DAPI (Vector Laboratories, Burlingame, Calif.). Epifluorescence images were taken using a Leica DM6000B microscope equipped with a CCD camera (Leica DFC350Fx) and FW4000 software. The primary antibodies for immunocytochemistry were as follows: phospho-STAT3 (Tyr705, 1:50, Cell signaling, Danvers, Mass.); α-sarcomeric actin (1:500, Sigma); α-smooth muscle actin (1:800, Sigma); and von Willebrand factor (1:100, Chemicon, Temecula, Calif.). For quantification of differentiation, cells positive for α-sarcomeric actin, α-smooth muscle actin and von Willebrand factor and total cells were counted at least in three fields per slide. The percentage of positive cells was calculated for each slide (n=3 in each group).

### DNA Replication Assay.

Three days after the plating, cardiac stem cells were cultured in the growth medium, conditioned media or serum-free medium for 24 hours, and BrdU (BD Biosciences) was added at a final concentration of 10 μM. Immunocytochemistry with the use of BrdU antibody (Sigma) and quantification of BrdU-positive cells were performed as described above.

### Immunoblotting.

Cells were lysed in a buffer that consisted of 0.1% sodium dodecyl sulphate (SDS) and complete protease inhibitor cocktail (Roche, Basel, Switzerland) in PBS. Protein concentration was determined by the DC protein assay (Biorad, Hercules, Calif.). Twenty μg of protein was separated by SDS-PAGE. After electrophoresis, the gels were electroblotted to polyvinylidene difluoride (PVDF) membranes. All electrophoresis and electroblotting used Novex reagents and systems (Invitrogen, Carlsbad, Calif.). The blots were blocked for 1 h at RT in 5% nonfat dry milk in PBS with 0.1% Tween 20 (PBST), washed 3×5 min in PBST, and incubated in primary antibodies in PBST with 5% BSA overnight at 4° C. After 3×5 minute washes in PBST, the blots were incubated in secondary antibody conjugated to horseradish peroxidase conjugate (1:2000, Sigma) in PBST for 1 h our at room temperature. Unbound secondary antibody was removed and positive bands were detected with a chemiluminescent reaction. The primary antibodies for immunoblotting were Ki67 (clone SP6, 1:200, Abeam, Cambridge, Mass.); phospho-STAT3 (1:1000); total STAT3 (1:1000, Cell signaling); and β-actin (1:5000, Sigma).

### ELISAs

Concentrations of adrenomedullin, hepatocyte growth factor (HGF), LIF, stromal-derived factor-1 (SDF-1), vascular endothelial growth factor (VEGF), and Dickkopf-1 were measured in CdM by ELISA according to the instructions of the manufacturer (adrenomedullin, Phoenix Pharmaceuticals, Burlingame, Calif.; HGF, IL-6, LIF, SDF-1, VEGF, Dickkopf-1, R&D systems, Minneapolis, Minn.).

### Statistical Analysis.

All values are expressed as mean±SEM unless otherwise indicated.

Comparisons of parameters among the three groups were made using one-way analysis of variance (ANOVA) followed by Scheffé's multiple comparison test. Comparisons of parameters between two groups were made by unpaired Student's t-test. P <0.05 was considered significant.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A cellular composition comprising an isolated bone marrow-derived cell that expresses CD133 or CD271/p75-low affinity nerve growth factor receptor.

2. The composition of claim 1, wherein the cell is an in vitro-derived progeny cell of a bone marrow derived cell that expresses CD133 or CD271/p75-low affinity nerve growth factor receptor.

3. (canceled)

4. (canceled)

5. The composition of claim 1, wherein the cellular composition comprises cells that express one or more surface epitopes selected from the group consisting of CD133⁺, CD45⁺, CD34⁺, ABC G2⁺, CD24⁺, and fail to express detectable levels or express reduced levels of a surface epitope selected from the group consisting of CD49a, CD49b, CD90, and CD105.

6. The composition of claim 1, wherein the cellular composition comprises cells that at passage 2 fail to express detectable levels or express reduced levels of a surface epitope selected from the group consisting of CD133, CD45, CD34, CD31, ABCG2 or CD24.

7. The composition of claim 1, wherein the cellular composition comprises cells that express a surface epitope selected from the group consisting of CD90 (Thy 1), CD105 (Endoglin), CD29, CD44, CD59, CD49a and CD49b.

8-9. (canceled)

10. A composition comprising secreted cellular factors in a pharmaceutical excipient, wherein the cellular factors are
derived from a cell of claim 1.

11. A composition comprising secreted cellular factors in a pharmaceutical excipient, wherein the cellular factors are
greater than about 5 kD is size;
detectable in an immunoassay;
secreted by an isolated bone marrow-derived non-hematopoietic progenitor cell selected for expression of CD133 or CD271/p75-low affinity nerve growth factor receptor;
have a biological activity selected from the group consisting of reducing cell death in a cell population at risk thereof, increasing cell survival, reducing inflammation, increase cell proliferation; and
inactivated by heat denaturation.]

12. A method for generating a composition that promotes tissue repair, the method comprising:
(a) selecting an isolated bone marrow-derived cell that expresses CD133 or CD271/p75-low affinity nerve growth factor receptor; and
(b) incubating the cell in growth media to enrich said media for cell-secreted factors, thereby generating a composition that promotes tissue repair.

13. The method of claim 12, wherein the method further comprises (c) purifying the cell-secreted factors.

14-18. (canceled)

19. A method for increasing cell survival or proliferation, the method comprising
(a) obtaining a composition according to the method of claim 12, and
(b) contacting a cell at risk of cell death with the composition, thereby increasing cell survival or proliferation.

20. The method of claim 19, wherein the method stabilizes or reduces tissue damage in a subject.

21. The method of claim 19, wherein the, composition comprises factors secreted by an isolated bone marrow-derived cell that expresses CD133 or CD271/p75-low affinity nerve growth factor receptor.

22-27. (canceled)

28. The method of claim 20, wherein the subject has a disease selected from the group consisting of myocardial infarction, congestive heart failure, stroke, ischemia, and wound healing.

29. The method of claim 20, wherein the method improves motor function after stroke or improves heart function after an ischemic event relative to the subject's function prior to treatment or relative to a reference.

30. (canceled)

31. The composition of claim 11, wherein the composition is a subject-specific cellular composition.

32-40. (canceled)

41. A method for treating or preventing ischemic damage in a subject, the method comprising contacting a cell at risk of ischemic injury with an effective amount of a composition comprising factors secreted by an isolated bone marrow-derived cell that expresses CD133 or CD271/p75-low affinity nerve growth factor receptor; thereby increasing cell survival or proliferation.

42. The method of claim 41, wherein the factors are derived from a cell is isolated from said subject.

43-57. (canceled)

58. A method for identifying an agent useful for tissue repair or regeneration:
contacting a cell or cell population at risk of cell death with a composition of agents secreted by an isolated bone marrow-derived non-hematopoietic progenitor cell selected for expression of CD133 and/or CD271/p75-low affinity nerve growth factor receptor;
detecting an increase in cell survival, growth, or proliferation or a decrease in cell death relative to an untreated control cell or cell population.
identifying an agent or fraction of the composition that reduces cell death, increases cell growth or proliferation.
